SEQUENCE LISTING (183 sequences)

SEQ ID NO: 1 - RNA, 19 nt, Artificial sequence
  Synthetic polynucleotide
  misc_feature: ON-TARGETplus human NAPRT siRNA 1, Catalog Number L016912010005, Clone ID L01691209, Dharmacon
gcaacaacauugacgagga

SEQ ID NO: 2 - RNA, 19 nt, Artificial sequence
  Synthetic polynucleotide
  misc_feature: ON-TARGETplus human NAPRT siRNA 2, Catalog Number L016912010005, Clone ID L01691210, Dharmacon
cuggaaacaacacgaauca

SEQ ID NO: 3 - RNA, 19 nt, Artificial sequence
  Synthetic polynucleotide
  misc_feature: ON-TARGETplus human NAPRT siRNA 3, Catalog Number L016912010005, Clone ID L01691211, Dharmacon
gguagagcccugacuggga

SEQ ID NO: 4 - RNA, 19 nt, Artificial sequence
  Synthetic polynucleotide
  misc_feature: ON-TARGETplus human NAPRT siRNA 4, Catalog Number L016912010005, Clone ID L01691212, Dharmacon
ggacaguggugaccugcua

SEQ ID NO: 5 - DNA, 21 bp, Artificial sequence
  Synthetic polynucleotide
  misc_feature: Silencer Select human NAPRT siRNA 5, Catalog Number L016912010005, Clone ID s195572, Ambion ThermoFischer Scientific
gaaugaagcugaccgaggatt

SEQ ID NO: 6 - RNA, 19 nt, Artificial sequence
  Synthetic polynucleotide
  misc_feature: ON-TARGETplus human NAMPT siRNA 1, Catalog Number L004581000005, Clone ID J00458105, Dharmacon
gguaagaaguuuccuguua

SEQ ID NO: 7 - RNA, 19 nt, Artificial sequence
  Synthetic polynucleotide
  misc_feature: ON-TARGETplus human NAMPT siRNA 2, Catalog Number L004581000005, Clone ID J00458106, Dharmacon
caaauuggauugagacuau

SEQ ID NO: 8 - RNA, 19 nt, Artificial sequence
  Synthetic polynucleotide
  misc_feature: ON-TARGETplus human NAMPT siRNA 3, Catalog Number L004581000005, Clone ID J00458107, Dharmacon
uaacuuagauggucuggaa

SEQ ID NO: 9 - RNA, 19 nt, Artificial sequence
  Synthetic polynucleotide
  misc_feature: ON-TARGETplus human NAMPT siRNA 4, Catalog Number L004581000005, Clone ID J00458108, Dharmacon
caagcaaaguuuauuccua

SEQ ID NO: 10 - DNA, 21 bp, Artificial sequence
  Synthetic polynucleotide
  misc_feature: Silencer Select human NAMPT siRNA 1, Catalog Number L004581000005, Clone ID s19725, Ambion ThermoFischer Scientific
caaauuggauugagacuautt

SEQ ID NO: 11 - RNA, 19 nt, Artificial sequence
  Synthetic polynucleotide
  misc_feature: ON-TARGETplus human NADSYN1 siRNA 1, Catalog Number L007723010005, Clone ID J00772309, Dharmacon
ggaaauaugcggcuacgga

SEQ ID NO: 12 - RNA, 19 nt, Artificial sequence
  Synthetic polynucleotide
  misc_feature: ON-TARGETplus human NADSYN1 siRNA 2, Catalog Number L007723010005, Clone ID J00772310, Dharmacon
cauuauuacgagucggaca

SEQ ID NO: 13 - RNA, 19 nt, Artificial sequence
  Synthetic polynucleotide
  misc_feature: ON-TARGETplus human NADSYN1 siRNA 3, Catalog Number L007723010005, Clone ID J00772311, Dharmacon
ucugcaaacuccucggcau

SEQ ID NO: 14 - RNA, 19 nt, Artificial sequence
  Synthetic polynucleotide
  misc_feature: ON-TARGETplus human NADSYN1 siRNA 4, Catalog Number L007723010005, Clone ID J00772312, Dharmacon
cugacaaagugaagcgguu

SEQ ID NO: 15 - DNA, 21 bp, Artificial sequence
  Synthetic polynucleotide
  misc_feature: Silencer Select human NADSYN1 siRNA 5, Catalog Number L007723010005, Clone ID s30396, Dharmacon
ggguggaucucgugacuautt

SEQ ID NO: 16 - RNA, 19 nt, Artificial sequence
  Synthetic polynucleotide
  misc_feature: ON-TARGETplus human NMRK1 siRNA 1, Catalog Number L006722010005, Clone ID J00672209, Dharmacon
cuccagacucuccgggaua

SEQ ID NO: 17 - RNA, 19 nt, Artificial sequence
  Synthetic polynucleotide
  misc_feature: ON-TARGETplus human NMRK1 siRNA 2, Catalog Number L006722010005, Clone ID J00672210, Dharmacon
ggacuguaccuuagcaagu

SEQ ID NO: 18 - RNA, 19 nt, Artificial sequence
  Synthetic polynucleotide
  misc_feature: ON-TARGETplus human NMRK1 siRNA 3, Catalog Number L006722010005, Clone ID J00672211, Dharmacon
cuuucugaacauagcucua

SEQ ID NO: 19 - RNA, 21 nt, Artificial sequence
  Synthetic polynucleotide
  misc_feature: ON-TARGETplus human NMRK1 siRNA 4, Catalog Number L006722010005, Clone ID J00672212, Dharmacon
cuugacacuauauauggaaua

SEQ ID NO: 20 - DNA, 21 bp, Artificial sequence
  Synthetic polynucleotide
  misc_feature: Silencer Select human NMRK1 siRNA 5, Catalog Number L006722010005, Clone ID s29916, Dharmacon
gacacuauauggaauagaatt

SEQ ID NO: 21 - RNA, 19 nt, Artificial sequence
  Synthetic polynucleotide
  misc_feature: ON-TARGETplus human NMRK2 siRNA 1, Catalog Number L006747010005, Clone ID J00674710, Dharmacon
uggucuaccuggacggcau

SEQ ID NO: 22 - RNA, 19 nt, Artificial sequence
  Synthetic polynucleotide
  misc_feature: ON-TARGETplus human NMRK2 siRNA 2, Catalog Number L006747010005, Clone ID J00674711, Dharmacon
ggacagcaugugagcguuu

SEQ ID NO: 23 - RNA, 19 nt, Artificial sequence
  Synthetic polynucleotide
  misc_feature: ON-TARGETplus human NMRK2 siRNA 3, Catalog Number L006747010005, Clone ID J00674712, Dharmacon
gggacgugcuggagucucu

SEQ ID NO: 24 - RNA, 19 nt, Artificial sequence
  Synthetic polynucleotide
  misc_feature: ON-TARGETplus human NMRK2 siRNA 4, Catalog Number L006747010005, Clone ID J00674713, Dharmacon
cagaaguauaggcaggaga

SEQ ID NO: 25 - DNA, 21 bp, Artificial sequence
  Synthetic polynucleotide
  misc_feature: Silencer Select human NMRK2 siRNA 5, Catalog Number L006747010005, Clone ID s25996, Ambion ThermoFischer Scientific
acugcugcgugauccaucatt

SEQ ID NO: 26 - DNA, 21 bp, Artificial sequence
  Synthetic polynucleotide
  misc_feature: Silencer Select human STAT3 siRNA 1, Clone ID S744, Ambion ThermoFischer Scientific
ggcuggacaauaucauugatt

SEQ ID NO: 27 - DNA, 21 bp, Artificial sequence
  Synthetic polynucleotide
  misc_feature: Silencer Select human STAT3 siRNA 2, Clone ID S745, Ambion ThermoFischer Scientific
gcaccuuccugcuaagauutt

SEQ ID NO: 28 - RNA, 24 nt, Artificial sequence
  Synthetic polynucleotide
  misc_feature: Human GATA3 siRNA 1, Song H. JBC. 2009
gcagggcaguaucaugaagccuaa

SEQ ID NO: 29 - RNA, 24 nt, Artificial sequence
  Synthetic polynucleotide
  misc_feature: Human GATA3 siRNA 2, Song H. JBC. 2009
ggcaugaaggaugccaagaaguuu

SEQ ID NO: 30 - RNA, 19 nt, Artificial sequence
  Synthetic polynucleotide
  misc_feature: Human MAX siRNA, Babic. Cell Metabolism. 2013
caaagacagcuuucacagu

SEQ ID NO: 31 - DNA, 21 bp, Artificial sequence
  Synthetic polynucleotide
  misc_feature: Silencer Select Human MYC siRNA, Ambion ThermoFischer Scientific
gagcuaaaacggagcuuuutt

SEQ ID NO: 32 - RNA, 19 nt, Artificial sequence
  Synthetic polynucleotide
  misc_feature: Human FOXM1 siRNA, Wu. Acta Pharmacologica Sinica. 2010
ggaccacuuucccuacuuu

SEQ ID NO: 33 - DNA, 19 bp, Artificial sequence
  Synthetic polynucleotide
  misc_feature: shLuciferase Control, Li. F. Cancer Cell. 2015
cttacgctgagtacttcga

SEQ ID NO: 34 - DNA, 58 bp, Artificial sequence
  Synthetic polynucleotide
  misc_feature: NAMPT MISSION shRNA 1, Catalog Number TRCN0000116177, CloneID NM18279011070s1c1
ccggccaccttatcttagagttattctcgagaataactctaagataaggtggtttttg

SEQ ID NO: 35 - DNA, 58 bp, Artificial sequence
  Synthetic polynucleotide
  misc_feature: NAMPT MISSION shRNA 2, Catalog Number TRCN0000116180, CloneID NM1827901579s1c1
ccgggtaacttagatggtctggaatctcgagattccagaccatctaagttactttttg

SEQ ID NO: 36 - DNA, 58 bp, Artificial sequence
  Synthetic polynucleotide
  misc_feature: NAPRT MISSION shRNA 1, Catalog Number TRCN0000242730, CloneID NM145201494s21c1
ccggcaccatggcgttgggctattgctcgagcaatagcccaacgccatggtgtttttg

SEQ ID NO: 37 - DNA, 58 bp, Artificial sequence
  Synthetic polynucleotide
  misc_feature: NAPRT MISSION shRNA 2, Catalog Number TRCN0000242731, CloneID NM14520141192s21c1
ccggttccctgggtggcgtctataactcgagttatagacgccacccagggaatttttg

SEQ ID NO: 38 - DNA, 58 bp, Artificial sequence
  Synthetic polynucleotide
  misc_feature: NAPRT MISSION shRNA 3, Catalog Number TRCN0000242729, CloneID NM14520141069s21c1
ccgggtcagtcctcatcgtagtcagctcgagctgactacgatgaggactgactttttg

SEQ ID NO: 39 - DNA, 59 bp, Artificial sequence
  Synthetic polynucleotide
  misc_feature: NMRK1 MISSION shRNA 1, Catalog Number TRCN0000160349, CloneID NM178811382s1c1
ccggcttgaagcacttaacatggaactcgagttccatgttaagtgcttcaagttttttg

SEQ ID NO: 40 - DNA, 58 bp, Artificial sequence
  Synthetic polynucleotide
  misc_feature: NMRK1 MISSION shRNA 2, Catalog Number TRCN0000360129, CloneID NM178812782s21c1
ccgggcaagtatatgaagatctaatctcgagattagatcttcatatacttgctttttg

SEQ ID NO: 41 - DNA, 58 bp, Artificial sequence
  Synthetic polynucleotide
  misc_feature: NMRK2 MISSION shRNA 1, Catalog Number TRCN0000359472, CloneID NM1706782679s21c1
ccggggagaagtacccgcaactacactcgagtgtagttgcgggtacttctcctttttg

SEQ ID NO: 42 - DNA, 58 bp, Artificial sequence
  Synthetic polynucleotide
  misc_feature: NMRK2 MISSION shRNA 2, Catalog Number TRCN0000359397, CloneID NM1706782645s21c1
ccggttcctgaccgtcccgtatgaactcgagttcatacgggacggtcaggaatttttg

SEQ ID NO: 43 - DNA, 58 bp, Artificial sequence
  Synthetic polynucleotide
  misc_feature: NADSYN1 MISSION shRNA 1, Catalog Number TRCN0000044508, CloneID NM01816141446s1c1
ccggccaccacatcagtctcaacatctcgagatgttgagactgatgtggtggtttttg

SEQ ID NO: 44 - DNA, 58 bp, Artificial sequence
  Synthetic polynucleotide
  misc_feature: NADSYN1 MISSION shRNA 2, Catalog Number TRCN0000232702, CloneID NM0181614276s21c1
ccggctacggatgttgggatcattactcgagtaatgatcccaacatccgtagtttttg

SEQ ID NO: 45 - DNA, 19 bp, Artificial sequence
  Synthetic polynucleotide
  misc_feature: SMARTvector Inducible Human NAPRT mEF1a-TurboRFP shRNA 1 UTR, Catalog Number V3SH1125510EG93100, CloneID VSH11252226366844
tttgagatcacctaaacac

SEQ ID NO: 46 - DNA, 19 bp, Artificial sequence
  Synthetic polynucleotide
  misc_feature: SMARTvector Inducible Human NAPRT mEF1a-TurboRFP shRNA 2, Catalog Number V3SH1125510EG93100, CloneID VSH11252225421625
gagaccactcctccacacg

SEQ ID NO: 47 - DNA, 19 bp, Artificial sequence
  Synthetic polynucleotide
  misc_feature: SMARTvector Inducible Human NAPRT mEF1a-TurboRFP shRNA 3, Catalog Number V3SH1125510EG93100, CloneID VSH11252225577913
cctgcgatcaagcgaagcc

SEQ ID NO: 48 - DNA, 19 bp, Artificial sequence
  Synthetic polynucleotide
  misc_feature: SMARTvector Inducible Human NMRK1 mEF1a-TurboRFP shRNA 1 UTR, Catalog Number V3SH1125510EG54981, CloneID VSH11252226740701
gagctctgctgtatctatg

SEQ ID NO: 49 - DNA, 19 bp, Artificial sequence
  Synthetic polynucleotide
  misc_feature: SMARTvector Inducible Human NMRK1 mEF1a-TurboRFP shRNA 2, Catalog Number V3SH1125510EG54981, CloneID VSH11252226475183
ctgattccaatgataaatg

SEQ ID NO: 50 - DNA, 19 bp, Artificial sequence
  Synthetic polynucleotide
  misc_feature: SMARTvector Inducible Human NMRK1 mEF1a-TurboRFP shRNA 3, Catalog Number V3SH1125510EG54981, CloneID VSH1125222699253
tccgtctttatgctgtcac

SEQ ID NO: 51 - DNA, 19 bp, Artificial sequence
  Synthetic polynucleotide
  misc_feature: SMARTvector Inducible Human NMRK2 mEF1a-TurboRFP shRNA 1, Catalog Number V3SH1125510EG27231, CloneID VSH11252224727305
tgggcacgggcaaacttct

SEQ ID NO: 52 - DNA, 19 bp, Artificial sequence
  Synthetic polynucleotide
  misc_feature: SMARTvector Inducible Human NMRK2 mEF1a-TurboRFP shRNA 2, Catalog Number V3SH1125510EG27231, CloneID VSH11252225138627
gctgtagagcaggaagcct

SEQ ID NO: 53 - DNA, 19 bp, Artificial sequence
  Synthetic polynucleotide
  misc_feature: SMARTvector Inducible Human NMRK2 mEF1a-TurboRFP shRNA 3, Catalog Number V3SH1125510EG27231, CloneID VSH11252227090666
ttacagaggcgtcttgggg

SEQ ID NO: 54 - DNA, 19 bp, Artificial sequence
  Synthetic polynucleotide
  misc_feature: SMARTvector Inducible Human NAMPT mEF1a-TurboRFP shRNA 1 UTR, Catalog Number V3SH1125510EG10135, CloneID VSH11252229940249
ggaaaagtgagtgattcgt

SEQ ID NO: 55 - DNA, 19 bp, Artificial sequence
  Synthetic polynucleotide
  misc_feature: SMARTvector Inducible Human NAMPT mEF1a-TurboRFP shRNA 2, Catalog Number V3SH1125510EG10135, CloneID VSH11252226167755
caaacctccaccagaaccg

SEQ ID NO: 56 - DNA, 19 bp, Artificial sequence
  Synthetic polynucleotide
  misc_feature: SMARTvector Inducible Human NAMPT mEF1a-TurboRFP shRNA 3, Catalog Number V3SH1125510EG10135, CloneID VSH11252226214912
ttcaccccatattttctca

SEQ ID NO: 57 - DNA, 19 bp, Artificial sequence
  Synthetic polynucleotide
  misc_feature: SMARTvector Inducible Human NADSYN1 mEF1a-TurboRFP shRNA 1, Catalog Number V3SH1125508EG55191, CloneID VSH11252224734628
ctgagtttcccatagaccg

SEQ ID NO: 58 - DNA, 19 bp, Artificial sequence
  Synthetic polynucleotide
  misc_feature: SMARTvector Inducible Human NADSYN1 mEF1a-TurboRFP shRNA 2, Catalog Number V3SH1125508EG55191, CloneID VSH11252224850260
ggcaggtccaaggcttatc

SEQ ID NO: 59 - DNA, 19 bp, Artificial sequence
  Synthetic polynucleotide
  misc_feature: SMARTvector Inducible Human NADSYN1 mEF1a-TurboRFP shRNA 3, Catalog Number V3SH1125508EG55191, CloneID VSH11252225184913
ccgagaccagaggctcaac

SEQ ID NO: 60 - DNA, 21 bp, Artificial sequence
  Synthetic polynucleotide
  misc_feature: TBP primer 5′ to 3′
gagctgtgatgtgaagtttcc

SEQ ID NO: 61 - DNA, 22 bp, Artificial sequence
  Synthetic polynucleotide
  misc_feature: TBP primer 3′ to 5′
tctgggtttgatcattctgtag

SEQ ID NO: 62 - DNA, 21 bp, Artificial sequence
  Synthetic polynucleotide
  misc_feature: B2M primer 5′ to 3′
atgagtatgcctgccgtgtga

SEQ ID NO: 63 - DNA, 20 bp, Artificial sequence
  Synthetic polynucleotide
  misc_feature: B2M primer 3′ to 5′
ggcatcttcaaacctccatg

SEQ ID NO: 64 - DNA, 21 bp, Artificial sequence
  Synthetic polynucleotide
  misc_feature: YWHAZ primer 5′ to 3′
tgatccccaatgcttcacaag

SEQ ID NO: 65 - DNA, 23 bp, Artificial sequence
  Synthetic polynucleotide
  misc_feature: YWHAZ primer 3′ to 5′
gccaagtaacggtagtaatctcc

SEQ ID NO: 66 - DNA, 19 bp, Artificial sequence
  Synthetic polynucleotide
  misc_feature: RPL13A primer 5′ to 3′
ggaccgtgcgaggtatgct

SEQ ID NO: 67 - DNA, 21 bp, Artificial sequence
  Synthetic polynucleotide
  misc_feature: RPL13A primer 3′ to 5′
atgccgtcaaacaccttgaga

SEQ ID NO: 68 - DNA, 20 bp, Artificial sequence
  Synthetic polynucleotide
  misc_feature: NAMPT primer 1, 5′ to 3′
agtggtgcctgtgtacttct

SEQ ID NO: 69 - DNA, 20 bp, Artificial sequence
  Synthetic polynucleotide
  misc_feature: NAMPT primer 1, 3′ to 5′
tgcctgtatctgtggtcagc

SEQ ID NO: 70 - DNA, 22 bp, Artificial sequence
  Synthetic polynucleotide
  misc_feature: NAMPT primer 2, 5′ to 3′
aatgttctcttcacggtggaaa

SEQ ID NO: 71 - DNA, 21 bp, Artificial sequence
  Synthetic polynucleotide
  misc_feature: NAMPT primer 2, 3′ to 5′
actgtgattggataccaggac

SEQ ID NO: 72 - DNA, 22 bp, Artificial sequence
  Synthetic polynucleotide
  misc_feature: NAMPT primer 3, 5′ to 3′
atcctgttccaggctattctgt

SEQ ID NO: 73 - DNA, 22 bp, Artificial sequence
  Synthetic polynucleotide
  misc_feature: NAMPT 3 primer 3′ to 5′
ccccatattttctcacacgcat

SEQ ID NO: 74 - DNA, 20 bp, Artificial sequence
  Synthetic polynucleotide
  misc_feature: NMRK1 primer 1, 5′ to 3′
tcctcagcactttcctggtc

SEQ ID NO: 75 - DNA, 20 bp, Artificial sequence
  Synthetic polynucleotide
  misc_feature: NMRK primer 1, 3′ to 5′
cagccatttcctgctggatg

SEQ ID NO: 76 - DNA, 24 bp, Artificial sequence
  Synthetic polynucleotide
  misc_feature: NMRK1 primer 2, 5′ to 3′
tcatcctgagatatgacactgcaa

SEQ ID NO: 77 - DNA, 20 bp, Artificial sequence
  Synthetic polynucleotide
  misc_feature: NMRK1 primer 2, 3′ to 5′
gtggcaaaacaacactggct

SEQ ID NO: 78 - DNA, 25 bp, Artificial sequence
  Synthetic polynucleotide
  misc_feature: NMRK1 primer 3, 5′ to 3′
acacttttgctttgctagttcttgt

SEQ ID NO: 79 - DNA, 23 bp, Artificial sequence
  Synthetic polynucleotide
  misc_feature: NMRK1 primer 3, 3′ to 5′
tggaacaaaatctgaagaggacc

SEQ ID NO: 80 - DNA, 18 bp, Artificial sequence
  Synthetic polynucleotide
  misc_feature: NMRK2 primer 1, 5′ to 3′
atgaccaacggcggcaag

SEQ ID NO: 81 - DNA, 20 bp, Artificial sequence
  Synthetic polynucleotide
  misc_feature: NMRK2 primer 1, 3′ to 5′
atcctgatggatcacgcagc

SEQ ID NO: 82 - DNA, 19 bp, Artificial sequence
  Synthetic polynucleotide
  misc_feature: NMRK2 primer 2, 5′ to 3′
tctctggacatggaggcca

SEQ ID NO: 83 - DNA, 20 bp, Artificial sequence
  Synthetic polynucleotide
  misc_feature: NMRK2 primer 2, 3′ to 5′
agctgtagagcaggaagcct

SEQ ID NO: 84 - DNA, 20 bp, Artificial sequence
  Synthetic polynucleotide
  misc_feature: NMRK2 primer 3, 5′ to 3′
catgaagtcccgagaggagc

SEQ ID NO: 85 - DNA, 20 bp, Artificial sequence
  Synthetic polynucleotide
  misc_feature: NMRK2 primer 3, 3′ to 5′
gattcctgggagcggttcag

SEQ ID NO: 86 - DNA, 19 bp, Artificial sequence
  Synthetic polynucleotide
  misc_feature: NAPRT primer 1, 5′ to 3′
gtgaggtgaatgtcattgg

SEQ ID NO: 87 - DNA, 18 bp, Artificial sequence
  Synthetic polynucleotide
  misc_feature: NAPRT primer 1, 3′ to 5′
ggccaccagcttatagac

SEQ ID NO: 88 - DNA, 21 bp, Artificial sequence
  Synthetic polynucleotide
  misc_feature: NAPRT primer 2, 5′ to 3′
gccaatgacattcacctcact

SEQ ID NO: 89 - DNA, 17 bp, Artificial sequence
  Synthetic polynucleotide
  misc_feature: NAPRT primer 2, 3′ to 5′
aggtcttccgagctgct

SEQ ID NO: 90 - DNA, 20 bp, Artificial sequence
  Synthetic polynucleotide
  misc_feature: NAPRT primer 3, 5′ to 3′
tggacatgctgcagttagca

SEQ ID NO: 91 - DNA, 19 bp, Artificial sequence
  Synthetic polynucleotide
  misc_feature: NAPRT primer 3, 3′ to 5′
gcagtagtggctccacctg

SEQ ID NO: 92 - DNA, 20 bp, Artificial sequence
  Synthetic polynucleotide
  misc_feature: NADSYN1 primer 1, 5′ to 3′
cgcatactgaccacctgcta

SEQ ID NO: 93 - DNA, 20 bp, Artificial sequence
  Synthetic polynucleotide
  misc_feature: NADSYN1 primer 1, 3′ to 5′
caatctgctgggccaactct

SEQ ID NO: 94 - DNA, 20 bp, Artificial sequence
  Synthetic polynucleotide
  misc_feature: NADSYN1 primer 2, 5′ to 3′
ctcaacatcgatccagccgt

SEQ ID NO: 95 - DNA, 20 bp, Artificial sequence
  Synthetic polynucleotide
  misc_feature: NADSYN1 primer 2, 3′ to 5′
caaacagagggctcttcccc

SEQ ID NO: 96 - DNA, 20 bp, Artificial sequence
  Synthetic polynucleotide
  misc_feature: NADSYN1 primer 3, 5′ to 3′
acaagatgaccacgctcaca

SEQ ID NO: 97 - DNA, 21 bp, Artificial sequence
  Synthetic polynucleotide
  misc_feature: NADSYN1 primer 3, 3′ to 5′
atggtcgcagatcaaacctgt

SEQ ID NO: 98 - DNA, 20 bp, Artificial sequence
  Synthetic polynucleotide
  misc_feature: RPPH1 primer 1, 5′ to 3′
cggagggaagctcatcagtg

SEQ ID NO: 99 - DNA, 20 bp, Artificial sequence
  Synthetic polynucleotide
  misc_feature: RPPH1 primer 1, 3′ to 5′
tggccctagtctcagacctt

SEQ ID NO: 100 - DNA, 24 bp, Artificial sequence
  Synthetic polynucleotide
  misc_feature: HBB primer 1, 5′ to 3′
caggtacggctgtcatcacttaga

SEQ ID NO: 101 - DNA, 24 bp, Artificial sequence
  Synthetic polynucleotide
  misc_feature: HBB primer 1, 3′ to 5′
catggtgtctgtttgaggttgcta

SEQ ID NO: 102 - DNA, 24 bp, Artificial sequence
  Synthetic polynucleotide
  misc_feature: NAMPT primer 1, 5′ to 3′
aagtgaattttgagtctgggaaga

SEQ ID NO: 103 - DNA, 22 bp, Artificial sequence
  Synthetic polynucleotide
  misc_feature: NAMPT primer 1, 3′ to 5′
atcagagttggcaagaggaagc

SEQ ID NO: 104 - DNA, 22 bp, Artificial sequence
  Synthetic polynucleotide
  misc_feature: NAMPT primer 2, 5′ to 3′
aggcagtttggaagaggtagtg

SEQ ID NO: 105 - DNA, 20 bp, Artificial sequence
  Synthetic polynucleotide
  misc_feature: NAMPT primer 2, 3′ to 5′
tatgaggcgtccggaataga

SEQ ID NO: 106 - DNA, 20 bp, Artificial sequence
  Synthetic polynucleotide
  misc_feature: NAMPT primer 3, 5′ to 3′
ttaagtcactgctcggtcgg

SEQ ID NO: 107 - DNA, 18 bp, Artificial sequence
  Synthetic polynucleotide
  misc_feature: NAMPT primer 3, 3′ to 5′
ccagagctcccagactgc

SEQ ID NO: 108 - DNA, 22 bp, Artificial sequence
  Synthetic polynucleotide
  misc_feature: NMRK1 primer 1, 5′ to 3′
acaactcacaaagatggcttca

SEQ ID NO: 109 - DNA, 23 bp, Artificial sequence
  Synthetic polynucleotide
  misc_feature: NMRK1 primer 1, 3′ to 5′
ggccccttgacactatatggaat

SEQ ID NO: 110 - DNA, 23 bp, Artificial sequence
  Synthetic polynucleotide
  misc_feature: NMRK1 primer 2, 5′ to 3′
caggtctgcaatgccttcacttt

SEQ ID NO: 111 - DNA, 22 bp, Artificial sequence
  Synthetic polynucleotide
  misc_feature: NMRK1 primer 2, 3′ to 5′
atagaacagctatggagggggc

SEQ ID NO: 112 - DNA, 20 bp, Artificial sequence
  Synthetic polynucleotide
  misc_feature: NMRK1 primer 3, 5′ to 3′
tgctgtatctatgcgctccc

SEQ ID NO: 113 - DNA, 21 bp, Artificial sequence
  Synthetic polynucleotide
  misc_feature: NMRK1 primer 3, 3′ to 5′
actgtggtacaatgacagcca

SEQ ID NO: 114 - DNA, 20 bp, Artificial sequence
  Synthetic polynucleotide
  misc_feature: NMRK2 primer 1, 5′ to 3′
ggcgccaggttttctcaatg

SEQ ID NO: 115 - DNA, 19 bp, Artificial sequence
  Synthetic polynucleotide
  misc_feature: NMRK2 primer 1, 3′ to 5′
ggacgacttccaaggcagc

SEQ ID NO: 116 - DNA, 20 bp, Artificial sequence
  Synthetic polynucleotide
  misc_feature: NMRK2 primer 2, 5′ to 3′
ttccttgacgccacgagaac

SEQ ID NO: 117 - DNA, 20 bp, Artificial sequence
  Synthetic polynucleotide
  misc_feature: NMRK2 primer 2, 3′ to 5′
tcagcagccccacacttttc

SEQ ID NO: 118 - DNA, 20 bp, Artificial sequence
  Synthetic polynucleotide
  misc_feature: NMRK2 primer 3, 5′ to 3′
ccataggacacagagagcgg

SEQ ID NO: 119 - DNA, 21 bp, Artificial sequence
  Synthetic polynucleotide
  misc_feature: NMRK2 primer 3, 3′ to 5′
tcctggccagagagctcagat

SEQ ID NO: 120 - DNA, 20 bp, Artificial sequence
  Synthetic polynucleotide
  misc_feature: NAPRT primer 1, 5′ to 3′
ggtgctttgaaggtggacag

SEQ ID NO: 121 - DNA, 20 bp, Artificial sequence
  Synthetic polynucleotide
  misc_feature: NAPRT primer 1, 3′ to 5
cacgaatgaagctgaccgag

SEQ ID NO: 122 - DNA, 20 bp, Artificial sequence
  Synthetic polynucleotide
  misc_feature: NAPRT primer 2, 5′ to 3′
tcccagggtgaggatcacta

SEQ ID NO: 123 - DNA, 20 bp, Artificial sequence
  Synthetic polynucleotide
  misc_feature: NAPRT primer 2, 3′ to 5′
cttcagggtgtaaggagggg

SEQ ID NO: 124 - DNA, 20 bp, Artificial sequence
  Synthetic polynucleotide
  misc_feature: NAPRT primer 3, 5′ to 3′
cccaggtagctgtaggtgga

SEQ ID NO: 125 - DNA, 20 bp, Artificial sequence
  Synthetic polynucleotide
  misc_feature: NAPRT primer 3, 3′ to 5′
agagaagcggctgctagaga

SEQ ID NO: 126 - DNA, 21 bp, Artificial sequence
  Synthetic polynucleotide
  misc_feature: NADSYN1 primer 1, 5′ to 3′
gccctgctttccacatgacag

SEQ ID NO: 127 - DNA, 21 bp, Artificial sequence
  Synthetic polynucleotide
  misc_feature: NADSYN1 primer 1, 3′ to 5′
ccagctctgtcgcggttaatg

SEQ ID NO: 128 - DNA, 21 bp, Artificial sequence
  Synthetic polynucleotide
  misc_feature: NADSYN1 primer 2, 5′ to 3′
acatgacagtgtccccagagg

SEQ ID NO: 129 - DNA, 22 bp, Artificial sequence
  Synthetic polynucleotide
  misc_feature: NADSYN1 primer 2, 3′ to 5′
ttctcttcaaagcccttccagc

SEQ ID NO: 130 - DNA, 24 bp, Artificial sequence
  Synthetic polynucleotide
  misc_feature: NADSYN1 primer 3, 5′ to 3′
tctgatatcacaaagcttcgatcc

SEQ ID NO: 131 - DNA, 20 bp, Artificial sequence
  Synthetic polynucleotide
  misc_feature: NADSYN1 primer 3, 3′ to 5′
caggctcacgagacgatcag

SEQ ID NO: 132 - DNA, 43 bp, Artificial sequence
  Synthetic polynucleotide
  misc_feature: NAMPT Enhancer 4.6 Kb clone upstream, Primer ID PGL3Enh MLUIXHOI4.6K, 5′ to 3′
cgacgcgtaggattataaatcatgctgctataaagacacatgc

SEQ ID NO: 133 - DNA, 43 bp, Artificial sequence
  Synthetic polynucleotide
  misc_feature: NAMPT Enhancer 4.6 Kb clone upstream, Primer ID PGL3Enh MLUIXHOI4.6K, 3′ to 5′
cgctcgaggtcattacttacctgttggttgcatgcctcttaag

SEQ ID NO: 134 - DNA, 43 bp, Artificial sequence
  Synthetic polynucleotide
  misc_feature: NAMPT Enhancer 4.6 Kb clone downstream, Primer ID PGL3Enh XBAIXBAI4.6K, 5′ to 3′
cgtctagaaggattataaatcatgctgctataaagacacatgc

SEQ ID NO: 135 - DNA, 43 bp, Artificial sequence
  Synthetic polynucleotide
  misc_feature: NAMPT Enhancer 4.6 Kb clone downstream, Primer ID PGL3Enh XBAIXBAI4.6K, 3′ to 5′
cgtctagagtcattacttacctgttggttgcatgcctcttaag

SEQ ID NO: 136 - DNA, 34 bp, Artificial sequence
  Synthetic polynucleotide
  misc_feature: NAMPT Promoter, Primer ID PGL3proFW BGLIIHINDIII, 5′ to 3′
gtgagccgagatccagccactgcactccagcctg

SEQ ID NO: 137 - DNA, 40 bp, Artificial sequence
  Synthetic polynucleotide
  misc_feature: NAMPT Promoter, Primer ID PGL3proFW BGLIIHINDIII, 3′ to 5′
aagcttgcggggcgcggcagcgcgctgcgcagtgcgcgga

SEQ ID NO: 138 - DNA, 43 bp, Artificial sequence; identical to SEQ ID NO: 132
  Synthetic polynucleotide
  misc_feature: NAMPT Enhancer 4.6 Kb clone upstream A, Primer ID PGL3Enh MLUIXHOIA, 5′ to 3′

SEQ ID NO: 139 - DNA, 47 bp, Artificial sequence
  Synthetic polynucleotide
  misc_feature: NAMPT Enhancer 4.6 Kb clone upstream A, Primer ID PGL3Enh MLUIXHOIA, 3′ to 5′
ctcgagaccattatatgtggggctttcttagttacaacagctaggcc

SEQ ID NO: 140 - DNA, 43 bp, Artificial sequence
  Synthetic polynucleotide
  misc_feature: NAMPT Enhancer 4.6 Kb clone upstream B, Primer ID PGL3Enh MLUIXHOIB, 5′ to 3′
cgacgcgtgatgggagtttatttttctccatgtaataacccag

SEQ ID NO: 141 - DNA, 37 bp, Artificial sequence
  Synthetic polynucleotide
  misc_feature: NAMPT Enhancer 4.6 Kb clone upstream B, Primer ID PGL3Enh MLUIXHOIB, 3′ to 5′
ctcgagtagtttgttcattcatcactcattgggtgcc

SEQ ID NO: 142 - DNA, 39 bp, Artificial sequence
  Synthetic polynucleotide
  misc_feature: NAMPT Enhancer 4.6 Kb clone upstream C, Primer ID PGL3Enh MLUIXHOIC, 5′ to 3′
cgacgcgtgacctgaggctgttgacactcatagctttcc

SEQ ID NO: 143 - DNA, 39 bp, Artificial sequence
  Synthetic polynucleotide
  misc_feature: NAMPT Enhancer 4.6 Kb clone upstream C, Primer ID PGL3Enh MLUIXHOIC, 3′ to 5′
ctcgaggtatcatttgagccacattttaccatctcaccc

SEQ ID NO: 144 - DNA, 40 bp, Artificial sequence
  Synthetic polynucleotide
  misc_feature: NAMPT Enhancer 4.6 Kb clone upstream D, Primer ID PGL3Enh MLUIXHOID, 5′ to 3′
cgacgcgtagtaggaggattttccaggcctcacaggagtg

SEQ ID NO: 145 - DNA, 35 bp, Artificial sequence
  Synthetic polynucleotide
  misc_feature: NAMPT Enhancer 4.6 Kb clone upstream D, Primer ID PGL3Enh MLUIXHOID, 3′ to 5′
ctcgagactggggaggtgaaacacttcaaccatcc

SEQ ID NO: 146 - DNA, 33 bp, Artificial sequence
  Synthetic polynucleotide
  misc_feature: NAMPT Enhancer 4.6 Kb clone upstream E, Primer ID PGL3Enh MLUIXHOIE, 5′ to 3′
cgacgcgtctatcagtgcacaggctgtgcctgg

SEQ ID NO: 147 - DNA, 43 bp, Artificial sequence; identical to SEQ ID NO: 133
  Synthetic polynucleotide
  misc_feature: NAMPT Enhancer 4.6 Kb clone upstream E, Primer ID PGL3Enh MLUIXHOIE, 3′ to 5′

SEQ ID NO: 148 - DNA, 47 bp, Artificial sequence
  Synthetic polynucleotide
  misc_feature: NAMPT EnhA, Primer ID BCDE, GRCh37 hg19 DNA coordinates 105856198 105856822, 5′ to 3′
aggattataaatcatgctgcgcatagggtactggcctagctgttgta

SEQ ID NO: 149 - DNA, 47 bp, Artificial sequence
  Synthetic polynucleotide
  misc_feature: NAMPT EnhA, Primer ID BCDE, GRCh37 hg19 DNA coordinates 105856198 105856822, 3′ to 5′
tacaacagctaggccagtaccctatgcgcagcatgatttataatcct

SEQ ID NO: 150 - DNA, 54 bp, Artificial sequence
  Synthetic polynucleotide
  misc_feature: NAMPT EnhB, Primer ID ACDE, GRCh37 hg19 DNA coordinates 105856850 105857928, 5′ to 3′
gcatagggtactggcctagctgttgtacctttgctttacttggcaaagattagg

SEQ ID NO: 151 - DNA, 54 bp, Artificial sequence
  Synthetic polynucleotide
  misc_feature: NAMPT EnhB, Primer ID ACDE, GRCh37 hg19 DNA coordinates 105856850 105857928, 3′ to 5′
ctgtgaggcctggaaaatcctcctactcctaatctttgccaagtaaagcaaagg

SEQ ID NO: 152 - DNA, 54 bp, Artificial sequence
  Synthetic polynucleotide
  misc_feature: NAMPT EnhC, Primer ID ABDE, GRCh37 hg19 DNA coordinates 105857929 105858849, 5′ to 3′
cctttgctttacttggcaaagattaggagtaggaggattttccaggcctcacag

SEQ ID NO: 153 - DNA, 54 bp, Artificial sequence; identical to SEQ ID NO: 151
  Synthetic polynucleotide
  misc_feature: NAMPT EnhC, Primer ID ABDE, GRCh37 hg19 DNA coordinates 105857929 105858849, 3′ to 5′

SEQ ID NO: 154 - DNA, 52 bp, Artificial sequence
  Synthetic polynucleotide
  misc_feature: NAMPT EnhD, Primer ID ABCE, GRCh37 hg19 DNA coordinates 105858850 105859749, 5′ to 3′
agtaggaggattttccaggcctcacagctatcagtgcacaggctgtgcctgg

SEQ ID NO: 155 - DNA, 52 bp, Artificial sequence
  Synthetic polynucleotide
  misc_feature: NAMPT EnhD, Primer ID ABCE, GRCh37 hg19 DNA coordinates 105858850 105859749, 3′ to 5′
ccaggcacagcctgtgcactgatagctgtgaggcctggaaaatcctcctact

SEQ ID NO: 156 - DNA, 51 bp, Artificial sequence
  Synthetic polynucleotide
  misc_feature: NAMPT EnhE, Primer ID ABCD, GRCh37 hg19 DNA coordinates 105859750 105860665, 5′ to 3′
ctatcagtgcacaggctgtgcctgggctgtgtgtgcagtttgggtaagatc

SEQ ID NO: 157 - DNA, 51 bp, Artificial sequence
  Synthetic polynucleotide
  misc_feature: NAMPT EnhE, Primer ID ABCD, GRCh37 hg19 DNA coordinates 105859750 105860665, 3′ to 5′
gatcttacccaaactgcacacacagcccaggcacagcctgtgcactgatag

SEQ ID NO: 158 - DNA, 21 bp, Artificial sequence
  Synthetic polynucleotide
  misc_feature: NAMPT Enhancer Negative Control, Primer ID Neg, GRCh37 hg19 DNA coordinates 105843605 105843795, 5′ to 3′
actccagtctcaaagcctcat

SEQ ID NO: 159 - DNA, 21 bp, Artificial sequence
  Synthetic polynucleotide
  misc_feature: NAMPT Enhancer Negative Control, Primer ID Neg, GRCh37 hg19 DNA coordinates 105843605 105843795, 3′ to 5′
acccagagacagcatctgtaa

SEQ ID NO: 160 - DNA, 20 bp, Artificial sequence
  Synthetic polynucleotide
  misc_feature: NAMPT Enhancer H3K27ac, Primer ID P1, GRCh37 hg19 DNA coordinates 105857093 105857240, 5′ to 3′
cagctcattctcacccagca

SEQ ID NO: 161 - DNA, 20 bp, Artificial sequence
  Synthetic polynucleotide
  misc_feature: NAMPT Enhancer H3K27ac, Primer ID P1, GRCh37 hg19 DNA coordinates 105857093 105857240, 3′ to 5′
atggctacctgtccagcaac

SEQ ID NO: 162 - DNA, 20 bp, Artificial sequence
  Synthetic polynucleotide
  misc_feature: NAMPT Enhancer H3K27ac, Primer ID P2, GRCh37 hg19 DNA coordinates 105857229 105857462, 5′ to 3′
acaggtagccatctcagcca

SEQ ID NO: 163 - DNA, 20 bp, Artificial sequence
  Synthetic polynucleotide
  misc_feature: NAMPT Enhancer H3K27ac, Primer ID P2, GRCh37 hg19 DNA coordinates 105857229 105857462, 3′ to 5′
agctttctgggatcgggaag

SEQ ID NO: 164 - DNA, 25 bp, Artificial sequence
  Synthetic polynucleotide
  misc_feature: Dummy sgRNA F
caccgatcgtttccgcttaacggcg

SEQ ID NO: 165 - DNA, 25 bp, Artificial sequence
  Synthetic polynucleotide
  misc_feature: Dummy sgRNA R
aaaccgccgttaagcggaaacgatc

SEQ ID NO: 166 - DNA, 23 bp, Artificial sequence
  Synthetic polynucleotide
  misc_feature: g1 sgRNA target, DNA coordinates chr7 hg19 105791320 105791342
ctgcattggatggtccgtgtcgg

SEQ ID NO: 167 - DNA, 24 bp, Artificial sequence
  Synthetic polynucleotide
  misc_feature: g1 sgRNA F, DNA coordinates chr7 hg19 105791320 105791342
caccctgcattggatggtccgtgt

SEQ ID NO: 168 - DNA, 24 bp, Artificial sequence
  Synthetic polynucleotide
  misc_feature: g1 sgRNA R, DNA coordinates chr7 hg19 105791320 105791342
aaacacacggaccatccaatgcag

SEQ ID NO: 169 - DNA, 23 bp, Artificial sequence
  Synthetic polynucleotide
  misc_feature: g2 sgRNA target, DNA coordinates chr7 hg19 105856108 105856127
catgtccatcaatgatagactgg

SEQ ID NO: 170 - DNA, 24 bp, Artificial sequence
  Synthetic polynucleotide
  misc_feature: g2 sgRNA F, DNA coordinates chr7 hg19 105856108 105856127
cacccatgtccatcaatgatagac

SEQ ID NO: 171 - DNA, 24 bp, Artificial sequence
  Synthetic polynucleotide
  misc_feature: g2 sgRNA R, DNA coordinates chr7 hg19 105856108 105856127
aaacgtctatcattgatggacatg

SEQ ID NO: 172 - DNA, 23 bp, Artificial sequence
  Synthetic polynucleotide
  misc_feature: g3 sgRNA target, DNA coordinates chr7 hg19 105856983 105857005
aggatccaaagcaaccccgttgg

SEQ ID NO: 173 - DNA, 25 bp, Artificial sequence
  Synthetic polynucleotide
  misc_feature: g3 sgRNA F, DNA coordinates chr7 hg19 105856983 105857005
caccgaggatccaaagcaaccccgt

SEQ ID NO: 174 - DNA, 25 bp, Artificial sequence
  Synthetic polynucleotide
  misc_feature: g3 sgRNA R, DNA coordinates chr7 hg19 105856983 105857005
aaacacggggttgctttggatcctc

SEQ ID NO: 175 - DNA, 23 bp, Artificial sequence
  Synthetic polynucleotide
  misc_feature: g4 sgRNA target, DNA coordinates chr7 hg19 105857662 105857684
tccaagatcttcccggcatcggg

SEQ ID NO: 176 - DNA, 25 bp, Artificial sequence
  Synthetic polynucleotide
  misc_feature: g4 sgRNA F, DNA coordinates chr7 hg19 105857662 105857684
caccgtccaagatcttcccggcatc

SEQ ID NO: 177 - DNA, 25 bp, Artificial sequence
  Synthetic polynucleotide
  misc_feature: g4 sgRNA R, DNA coordinates chr7 hg19 105857662 105857684
aaacgatgccgggaagatcttggac

SEQ ID NO: 178 - DNA, 34 bp, Artificial sequence
  Synthetic polynucleotide
  misc_feature: nampt-FLAG, Primer ID NAMPT BamHI XmaI, 5′ to 3′
agagaattcggatccatgaatcctgcggcagaag

SEQ ID NO: 179 - DNA, 36 bp, Artificial sequence
  Synthetic polynucleotide
  misc_feature: nampt-FLAG, Primer ID NAMPT BamHI XmaI, 3′ to 5′
tggctcgagcccgggctaatgatgtgctgcttccag

SEQ ID NO: 180 - DNA, 46 bp, Artificial sequence
  Synthetic polynucleotide
  misc_feature: nmrk1-FLAG, Primer ID NMRK1 BamHI XmaI, 5′ to 3′
agagaattcggatccatgaaaacatttatcattggaatcagtggtg

SEQ ID NO: 181 - DNA, 37 bp, Artificial sequence
  Synthetic polynucleotide
  misc_feature: nmrk1-FLAG, Primer ID NMRK1 BamHI XmaI, 3′ to 5′
tggctcgagcccgggttatgctgtcacttgcaaacac

SEQ ID NO: 182 - DNA, 33 bp, Artificial sequence
  Synthetic polynucleotide
  misc_feature: naprt-FLAG, Primer ID NAPRT BamHI XmaI, 5′ to 3′
agagaattcggatccatggcggcggagcaggac

SEQ ID NO: 183 - DNA, 39 bp, Artificial sequence
  Synthetic polynucleotide
  misc_feature: naprt-FLAG, Primer ID NAPRT BamHI XmaI, 3′ to 5′
tggctcgagcccgggtcagggggactgccccgcacacag

(19) United States
(12) Patent Application Publication
(10) Pub. No.: US 2022/0133706 A1
(43) Pub. Date: May 5, 2022

(54) TARGETING NAD BIOSYNTHESIS IN THE TREATMENT OF CANCER

(71) Applicants: THE REGENTS OF THE UNIVERSITY OF CALIFORNIA, Oakland, CA (US); LUDWIG INSTITUTE FOR CANCER RESEARCH LTD, Zurich (CH)

(72) Inventors: Paul S. MISCHEL, La Jolla, CA (US); Bing REN, Del Mar, CA (US); Vineet BAFNA, San Diego, CA (US); Sudhir CHOWDHRY, San Diego, CA (US)

(21) Appl. No.: 17/438,610

(22) PCT Filed: Mar. 13, 2020
(86) PCT No.: PCT/US20/22812

§ 371 Date: Sep. 13, 2021

Related U.S. Application Data
(60) Provisional application No. 62/818,489, filed on Mar. 14, 2019.
(60) Provisional application No. 62/840,273, filed on Apr. 29, 2019.
(60) Provisional application No. 62/880,535, filed on Jul. 30, 2019.

(51) Int. Cl. A61K 31/4545; A61P 35/00; A61K 31/455; A61K 31/63; A61K 45/06

(52) U.S. Cl. CPC A61K 31/4545; A61P 35/00; A61K 45/06; A61K 31/63; A61K 31/455

(57) ABSTRACT

There are provided, inter alia, methods for treatment of cancer. The methods include means of identifying the NAD biosynthetic pathway on which depend cancer cells in a tumor, in a subject suffering from cancer. The methods further include administering to a subject in need a therapeutically effective amount of an inhibitor of the NMRK1 enzyme pathway to a tumor where the cancer cells are dependent of the NAD salvage pathway, allowing to lower the of dose FK866 administered to the subject. The methods also include administering to a subject in need a therapeutically effective amount of a bacterial inhibitor of NADSYN1 to a tumor where the cancer cells depend on the Preiss Handler pathway.

### CROSS-REFERENCE TO RELATED APPLICATION

This application claims priority to U.S. Provisional Application No. 62/818,489, filed Mar. 14, 2019, U.S. Provisional Application No. 62/840,273, filed Apr. 29, 2019, and U.S. Provisional Application No. 62/880,535, filed Jul. 30, 2019, the disclosures of which are incorporated herein in its entirety and for all purposes.

### STATEMENT AS TO RIGHTS TO INVENTIONS MADE UNDER FEDERALLY SPONSORED RESEARCH AND DEVELOPMENT

This invention was made with government support under GM114362 awarded by the National Institutes of Health. The government has certain rights in the invention.

### SEQUENCE LISTING

The instant application contains a Sequence Listing, which has been submitted electronically in ASCII format and is hereby incorporated by reference in its entirety. Said ASCII copy, created on Jul. 18, 2019 is named 048537-622P03US_SL_ST25 and is 54.3 KB in size.

### BACKGROUND

Precision oncology medicine involves linking tumor genotype with molecularly targeted drugs; however, targeting the frequently dysregulated metabolic landscape of cancer has proven to be a major challenge.

NAD (Nicotinamide adenine dinucleotide) is a critical small molecule co-factor in metabolic redox reactions, carrying high energy electrons to support oxidative phosphorylation by reversibly oxidizing or reducing NAD, and serving as a substrate for NAD-dependent enzymes that link cellular metabolism with epigenetic regulation and DNA damage repair. Mammalian cells make NAD by three different methods; (1) de novo synthesis from tryptophan; (2) generation from nicotinic acid using the Preiss-Handler (PH) pathway; or (3) synthesis from nicotinamide (NAM) or nicotinamide riboside via the salvage pathway. There remains a need for delineation of the molecular mechanisms that dictate NAD synthesis pathway choice and their role in cancer in order to use and develop cancer therapeutic targets. [See, for example, Refs. 1-14]

### BRIEF SUMMARY

In view of the foregoing, there remains a need for new agents and treatment strategies to improve survival for patients with cancer. Some embodiments of the present disclosure address this need, and/or provide additional benefits.

In an aspect, provided herein are methods for treating cancer in a subject in need thereof, including administering to the subject a therapeutically effective amount of an anticancer agent, where the subject has an amplified Preiss Handler pathway gene.

In an aspect, provided herein are methods for treating cancer in a subject in need thereof, including administering to the subject a therapeutically effective amount of an anticancer agent, where the subject has a Preiss Handler pathway-dependent cancer.

In an aspect, provided herein are methods for treating cancer in a subject in need thereof, including administering to the subject a therapeutically effective amount of an anticancer agent, where the subject has a NAD salvage pathway-dependent cancer.

In an aspect, provided herein are methods of detecting NAPRT gene expression or NADSYN1 gene expression in a subject that has cancer, including measuring in a biological sample from the subject, a DNA copy number of a gene selected from NAPRT and NADSYN1.

In an aspect, provided herein are methods of detecting NAPRT gene expression or NADSYN1 gene expression in a subject that has cancer, including measuring in a biological sample from the subject gene amplification or increased protein expression of NAPRT and NADSYN1.

In an aspect, provided herein is a composition including a NAMPT inhibitor and a NMRK1 inhibitor.

### BRIEF DESCRIPTION OF THE DRAWINGS

FIGS. 1A-F present data showing that PH-pathway survival addiction is not subject to enzymatic bypass, resulting in massive tumor cell death in vivo, whereas epigenetically determined dependence on the salvage pathway is subject to resistance by enzymatic bypass. FIG. 1A, top panels show line graphs depicting tumor volumes of nude mice bearing engineered OV4PH-amp and H460Sal-dep cells implanted subcutaneously into the left flank (top left panel) or right flank (top right panel). FIG. 1A, bottom panels show bar graphs depicting intratumoral NAD+ levels at Day 30: the left flank experiment (bottom left panel) and the right flank experiment (bottom right panel). FIG. 1B, left panel shows a line graph depicting tumor volume of nude mice bearing engineered H460Sal-dep cells implanted subcutaneously; FIG. 1B, right panel shows a bar graph depicting intratumoral NAD+ levels. FIG. 1C, top left panel shows the crystal structure of glutamine-dependent NAD synthetase from B. subtilis, bound to ATP and NAAD. Sequence alignment of P loop and NAAD-binding sites are shown. FIG. 1C, top right panel shows a line graph depicting NAD synthetase activity of human recombinant enzyme. FIG. 1C, middle left panel shows a line graph depicting cell viability of non-cancer and cancer cells treated with NADSYN1i for 72 hours. FIG. 1C, middle right panel shows a bar graph depicting the intracellular measurement of NAD+ levels in non-cancer and cancer cell lines (‘PH-amp’ and ‘Sal-dep’) treated with NADSYN1i and summarized in bottom chart. FIG. 1D, top panels shows line graphs depicting tumor volumes of nude mice bearing OV4PH-amp and H460Sal-dep cells implanted subcutaneously into the left flank (top left panel) or right flank (top right panel); mice were injected intraperitoneally (IP) with NADSYN1i once daily. FIG. 1D, bottom panels shows bar graphs depicting intratumoral NAD+ levels at Day 21: the left flank experiment (bottom left panel) and the right flank experiment (bottom right panel). FIG. 1E, top panel shows a line graph depicting tumor volume of nude mice bearing H460Sal-dep cells implanted subcutaneously into the right flank. Mice were injected intraperitoneally with FK-866 twice daily. FIG. 1E, bottom panel shows a bar graph depicting intratumoral NAD+ levels at Day 24. FIG. 1F shows a schematic overview of the molecular basis of NAD metabolic pathway addiction in cancer.

FIGS. 2A-E present data showing in vivo demonstration of NAD metabolic pathway dependencies. FIG. 2A shows a schematic overview of the experiment. OV4^{PH-amp} cells stably expressing DOX-inducible shRNA against NAPRT, NADSYN1, NAMPT, or NMRK1 were inoculated into the left flank of nude mice. H460^{Sal-dep} cells stably expressing DOX-inducible shRNAs were inoculated into the right flank of the same nude mice. ishNTC was used as a non-targeting control for both tumor types. FIG. 2B shows a line graph depicting tumor volume (left panel) and a bar graph depicting intratumoral NAD+ measurement (right panel) of nude mice bearing OV4^{PH-amp} cell stably expressing DOX-inducible shRNA against NMRK1 taken at the end of experiment on day 30. Tumor volume was monitored over a 30-day period. DOX treatment was initiated on day 7 after implantation until the end of the experiment. FIGS. 2C, 2D, show bar graphs depicting quantification of TUNEL+ nuclei and Ki67+ cells. DAPI was used to stain DNA for TUNEL staining. For TUNEL+ nuclei, 10,000-12,000 cells were counted for each cohort; for Ki67+ cells, 15,000-20,000 cells were counted for each cohort. FIG. 2E shows immunoblots for cleaved-caspase-3 as a measure of cell death and to test for protein abundance of NAMPT, NAPRT, NMRK1 and NADSYN1 in tumor tissues obtained from the indicated tumor types. Representative blots are from one of two independent experiments. Both biological replicates showed similar results. Actin was used as a loading control. Data are representative of eight (FIG. 2B left panel, FIGS. 2C, 2D) and five (FIG. 2B right panel) independent biological replicates from two independent experiments. Data are mean tumor volume±s.e.m. (n=8 tumors per cohort), with values determined by two-way ANOVA on repeated measurements over time (FIG. 2B, left panel). Data in scatter plots are mean±s.d., with P values determined by one-way ANOVA with Tukey's multiple comparisons test (FIGS. 2C, 2D. ns=not significant.

### DETAILED DESCRIPTION

### I. Definitions

Before embodiments of the present disclosure are further described, it is to be understood that this disclosure is not strictly limited to particular embodiments described, as such may of course vary. It is also to be understood that the terminology used herein is for the purpose of describing particular embodiments only, and is not intended to be limiting.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this disclosure belongs as understood in light of this disclosure. Although any methods and materials similar or equivalent to those described herein can also be used in the practice or testing of the present disclosure, the certain methods and materials are now described. All publications mentioned herein are incorporated herein by reference to disclose and describe the methods and/or materials in connection with which the publications are cited and are likewise incorporated by reference herein in their entirety. The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art in light of this disclosure. See, e.g., Singleton et al., DICTIONARY OF MICROBIOLOGY AND MOLECULAR BIOLOGY 2^{nd} ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this disclosure. The following descriptions are provided to facilitate understanding of certain terms used frequently herein and are not meant to limit the scope of the present disclosure.

As used herein, the term “nucleic acid” is used in accordance with its plain and ordinary meaning in light of the present disclosure and refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. The term “nucleic acid mimetic” is used in accordance with its plain and ordinary meaning in light of the present disclosure and refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs). As may be used herein, the terms “nucleic acid,” “nucleic acid molecule,” “nucleic acid oligomer,” “oligonucleotide,” “nucleic acid sequence,” “nucleic acid fragment” and “polynucleotide” may sometimes be used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. Nucleic acids, including nucleic acids with a phosphothioate backbone can include one or more reactive moieties. As used herein, the term “reactive moiety” is used in accordance with its plain and ordinary meaning in light of the present disclosure and includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, the term “gene” is used in accordance with its plain and ordinary meaning in light of the present disclosure and refers to the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

As used herein, the terms “expression” or “expressed” are used in accordance with their plain and ordinary meanings in light of the present disclosure and refer to the transcriptional and/or translational product of a gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

As used herein, the terms “plasmid” or “expression vector” are used in accordance with their plain and ordinary meanings in light of the present disclosure and refer to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “amino acid” is used in accordance with its plain and ordinary meaning in light of the present disclosure and refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” is used in accordance with its plain and ordinary meaning in light of the present disclosure and refers to chemical compounds that have a structure that is different from the general chemical structure of an amino acid, but that function in a manner similar to a naturally occurring amino acid.

As used herein, the term “conservatively modified variants” is used in accordance with its plain and ordinary meaning in light of the present disclosure and applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are generally conservative substitutions for one another: 1) Alanine (A), Glycine (G); 2) Aspartic acid (D), Glutamic acid I; 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, the terms “polypeptide,” “peptide” and “protein” are used in accordance with their plain and ordinary meanings in light of the present disclosure and refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, the term “isolated” is used in accordance with its plain and ordinary meaning in light of the present disclosure and, when applied to a nucleic acid or protein, refers to the nucleic acid or protein being essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

As used herein, the term “identical” or percent “identity”, are used in accordance with their plain and ordinary meanings in light of the present disclosure and in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the disclosure or individual domains of the polypeptides of the disclosure), when compared and aligned for maximum correspondence over a comparison window, or designated region as measured using one of the following sequence comparison algorithms or by manual alignment and visual inspection. Such sequences are then said to be “substantially identical.” This description also refers to the complement of a test sequence. Optionally, the identity exists over a region that is at least about 50 nucleotides in length, or over a region that is 100 to 500 or 1000 or more nucleotides in length.

As used herein, the term “percentage of sequence identity” is used in accordance with its plain and ordinary meaning in light of the present disclosure and is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

As used herein, the term “comparison window” is used in accordance with its plain and ordinary meaning in light of the present disclosure and includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art.

As used herein, the terms “complementary” or “complementarity” are used in accordance with their plain and ordinary meanings in light of the present disclosure and refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

As used herein, the term “stringent conditions” is used in accordance with its plain and ordinary meaning in light of the present disclosure and refers to hybridization conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part 1, Second Chapter “Overview of principles of hybridization and the strategy of nucleic acid probe assay”, Elsevier, N.Y.

As used herein, the term “hybridization” is used in accordance with its plain and ordinary meaning in light of the present disclosure and refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “contacting” is used in accordance with its plain ordinary meaning in light of the present disclosure and refers to the process of allowing at least two distinct species (e.g. nucleic acids and/or proteins) to become sufficiently proximal to react, interact or physically touch. It should be appreciated, that the resulting reaction product can be produced directly from a reaction between the added reagents or from an intermediate from one or more of the added reagents which can be produced in the reaction mixture.

As used herein, the terms “binding,” “specific binding” or “specifically binds” are used in accordance with their plain ordinary meanings in light of the present disclosure and refer to two or more molecules forming a complex that is relatively stable under physiologic conditions.

As used herein, the term “cell” is used in accordance with its plain ordinary meaning in light of the present disclosure and refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the terms “biological sample” or “sample” are used in accordance with their plain ordinary meanings in light of the present disclosure and refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

As used herein, the terms “control”, “control experiment” “control sample” and “control value” are used in accordance with their plain ordinary meanings in light of the present disclosure and refers to a sample, experiment, or value that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. A control experiment may be an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the terms “patient” or “subject in need thereof” are used in accordance with their plain ordinary meanings in light of the present disclosure and refer to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the terms “disease” or “condition” are used in accordance with their plain ordinary meanings in light of the present disclosure and refer to a state of being or health status of a patient or subject. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” is used in accordance with its plain ordinary meaning in light of the present disclosure and refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, Herceptin (trastuzumab) resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus, Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, lobular carcinoma, ductal carcinoma, cancer of the pancreatic stellate cells, cancer of the hepatic stellate cells, or prostate cancer. In embodiments, the cancer is selected from ovarian cancer, prostate cancer, esophageal cancer, salivary gland cancer, breast cancer, liver cancer, pancreatic cancer, stomach cancer, lung cancer, bladder cancer, colon cancer, and uterine cancer. In embodiments, the cancer is selected from muscle cancer, brain cancer, lymph node cancer, thyroid cancer, kidney cancer, and adrenal gland cancer.

As used herein, the terms “associated” or “associated with” are used in accordance with their plain ordinary meanings in light of the present disclosure and in the context of a substance or substance activity or function associated with a disease (e.g., cancer (e.g. leukemia, lymphoma, B cell lymphoma, or multiple myeloma)) means that the disease (e.g. cancer, (e.g. leukemia, lymphoma, B cell lymphoma, or multiple myeloma)) is caused by (in whole or in part), or a symptom of the disease is caused by (in whole or in part) the substance or substance activity or function.

As used herein, the terms “treatment” or “treating”, or “palliating” or “ameliorating” are used in accordance with their plain ordinary meanings in light of the present disclosure. These terms generally refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting or slowing the development of clinical symptoms by administration of a protective composition after their initial appearance; preventing re-occurring of the disease and/or relieving the disease, that is, causing the regression of clinical symptoms by administration of a protective composition after their initial appearance. In the disclosed methods, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90%, or greater as compared to a control level and such terms can include but do not necessarily include complete elimination.

“Treating” and “treatment” as used herein are used in accordance with their plain and ordinary meanings in light of the present disclosure and includes prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

As used herein, the term “prevent” is used in accordance with its plain and ordinary meaning in light of the present disclosure and refers to a decrease in the occurrence of disease symptoms in a patient. As indicated above, the prevention may be complete (no detectable symptoms) or partial, such that fewer symptoms are observed than would likely occur absent treatment.

As used herein, the term “effective amount” is used in accordance with its plain ordinary meaning in light of the present disclosure and refers to an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art in light of the present disclosure using known techniques in light of the present disclosure (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner in light of the present disclosure. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain ordinary meaning in light of the present disclosure and refers to oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the term “co-administer” is used in accordance with its plain ordinary meaning in light of the present disclosure and refers to a composition described herein administered at the same time, just prior to, or just after the administration of one or more additional therapies. The compounds of the disclosure can be administered alone or can be co-administered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

As used herein, the term “cancer model organism” is used in accordance with its plain ordinary meaning in light of the present disclosure and refers to an organism exhibiting a phenotype indicative of cancer, or the activity of cancer causing elements, within the organism. The term cancer is described above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

As used herein, the term “anticancer agent” is used in accordance with its plain ordinary meaning in light of the present disclosure and refers to a molecule (e.g. compound, peptide, protein, nucleic acid, antibody) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

As used herein, the term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

As used herein, the term “chimeric antibody” refers an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

As used herein, the term “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbone modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

As used herein, the terms “gene editing”, “genetic editing”, “genome editing”, or “genome engineering” refer to a type of genetic engineering in which DNA is inserted, deleted, modified or replaced in the genome of a living organism. Unlike early genetic engineering techniques that randomly insert genetic material into a host genome, genome editing targets the insertions to site specific locations. One key step in gene editing is creating a double stranded break at a specific point within a gene or genome. Examples of gene editing tools such as nucleases that accomplish this step include but are not limited to Zinc finger nucleases (ZFNs), transcription activator like effector nucleases (TALEN), meganucleases, and clustered regularly interspaced short palindromic repeats system (e.g. CRISPR/Cas).

As use herein the term “small molecule” within the fields of molecular biology and pharmacology, a small molecule is a low molecular weight (<900 daltons) organic compound that may regulate a biological process, with a size on the order of 1 nm. Many drugs are small molecules. Larger structures such as nucleic acids and proteins, and many polysaccharides are not small molecules, although their constituent monomers (ribo- or deoxyribonucleotides, amino acids, and monosaccharides, respectively) are often considered small molecules. Small molecules may be used as research tools to probe biological function as well as leads in the development of new therapeutic agents. Some can inhibit a specific function of a protein or disrupt protein-protein interactions. Pharmacology usually restricts the term “small molecule” to molecules that bind specific biological macromolecules and act as an effector, altering the activity or function of the target. Small molecules can have a variety of biological functions or applications, serving as cell signaling molecules, drugs in medicine, pesticides in farming, and in many other roles. These compounds can be natural (such as secondary metabolites) or artificial (such as antiviral drugs); they may have a beneficial effect against a disease (such as drugs) or may be detrimental (such as teratogens and carcinogens.

As used herein, the term “epigenetic inhibitor” is used in accordance with its plain ordinary meaning in light of the present disclosure and refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include vorinostat, romidepsin, CI-994, belinostat, panobinostat, givinostat, entinostat, mocetinostat, SRT501, CUDC-101, JNJ-26481585, or PCI24781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

As used herein, the term “multi-kinase inhibitor” is used in accordance with its plain ordinary meaning in light of the present disclosure and refers to a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Examples of anti-cancer multi-kinase inhibitors include dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

As used herein, the terms “PARP inhibitor”, “poly ADP ribose polymerase inhibitor” and “PARPi” is used in accordance with its plain ordinary meaning in light of the present disclosure and refers to a group of pharmacological inhibitors of the enzyme poly ADP ribose polymerase. They are developed for multiple indications, including the treatment of heritable cancers. Several forms of cancer are more dependent on PARP than regular cells, making PARP an attractive target for cancer therapy. Examples of PARP inhibitors include olaparib, rucaparib, niraparib, talazoparib, velaparib, and pamiparib. In embodiments, methods and compositions disclosed herein include combinations of a NAMPT inhibitor and a NMRK1 inhibitor with a PARP inhibitor.

As used herein, the terms “selective” and “selectivity” or the like of a compound are used in accordance with their plain ordinary meanings in light of the present disclosure and refer to the compound's ability to discriminate between molecular targets (e.g. a compound having selectivity toward HMT SUV39H1 and/or HMT G9a).

As used herein, the terms “specific”, “specifically”, “specificity”, or the like of a compound used in accordance with their plain ordinary meanings in light of the present disclosure and refer to the compound's ability to cause a particular action, such as inhibition, to a particular molecular target with minimal or no action to other proteins in the cell (e.g. a compound having specificity towards HMT SUV39H1 and/or HMT G9a displays inhibition of the activity of those HMTs whereas the same compound displays little-to-no inhibition of other HMTs such as DOT1, EZH1, EZH2, GLP, MLL1, MLL2, MLL3, MLL4, NSD2, SET1b, SET7/9, SET8, SETMAR, SMYD2, SUV39H2).

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one description of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the terms “inhibition”, “inhibit”, “inhibiting”, and the like in reference to a protein-inhibitor interaction are used in accordance with their plain and ordinary meanings in light of the present disclosure and mean negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

As used herein, the terms “inhibitor”, “repressor”, “antagonist”, or “downregulator” are used in accordance with their plain and ordinary meanings in light of the present disclosure and refer to a substance capable of detectably decreasing the expression or activity of a given gene or protein. The antagonist can decrease expression or activity 10%, 20%, 30%, 40%, 50%, 60%, 70%, 80%, 90%, or more in comparison to a control in the absence of the antagonist. In certain instances, expression or activity is 1.5-fold, 2-fold, 3-fold, 4-fold, 5-fold, 10-fold, or lower than the expression or activity in the absence of the antagonist.

For specific proteins described herein (e.g., NAMPT, NADSYN1, NAPRT, and the like), the named protein includes any of the protein's naturally occurring forms, or variants or homologs that maintain the protein transcription factor activity (e.g., within at least 50%, 80%, 90%, 95%, 96%, 97%, 98%, 99%, or 100% activity compared to the native protein). In some embodiments, variants or homologs have at least 90%, 95%, 96%, 97%, 98%, 99%, or 100% amino acid sequence identity across the whole sequence or a portion of the sequence (e.g. a 50, 100, 150, or 200 continuous amino acid portion) compared to a naturally occurring form. In embodiments, the protein is the protein as identified by its NCBI sequence reference. In embodiments, the protein is the protein as identified by its NCBI sequence reference or functional fragment or homolog thereof.

A “CRISPR associated protein 9,” “Cas9,” “Csn1”, or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99%, or 100% activity compared to Cas9). In some aspects, the variants or homologs have at least 90%, 95%, 96%, 97%, 98%, 99%, or 100% amino acid sequence identity across the whole sequence or a portion of the sequence (e.g. a 50, 100, 150, or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence. In embodiments, the CRISPR nucleic acid sequence is a prokaryotic nucleic acid sequence. In embodiments, the Cas9 nuclease from Streptococcus pyogenes is targeted to genomic DNA by a synthetic guide RNA consisting of a 20-nt guide sequence and a scaffold. The guide sequence base-pairs with the DNA target, directly upstream of a requisite 5′-NGG protospacer adjacent motif (PAM), and Cas9 mediates a double-stranded break (DSB) about 3-base pair upstream of the PAM. In embodiments, the CRISPR nuclease from Streptococcus aureus is targeted to genomic DNA by a synthetic guide RNA consisting of a 21-23-nt guide sequence and a scaffold. The guide sequence base-pairs with the DNA target, directly upstream of a requisite 5′-NNGRRT protospacer adjacent motif (PAM), and Cas9 mediates a double-stranded break (DSB) about 3-base pair upstream of the PAM.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) may generated in four sequential steps. First, two non-coding RNAs, the pre-crRNA array and tracrRNA, may be transcribed from the CRISPR locus. Second, tracrRNA may hybridize to the direct repeats of pre-crRNA, which is then processed into mature crRNAs containing individual spacer sequences. Third, the mature crRNA:tracrRNA complex may direct Cas9 to the DNA target consisting of the protospacer and the corresponding PAM via heteroduplex formation between the spacer region of the crRNA and the protospacer DNA. Finally, Cas9 may mediate cleavage of target DNA upstream of PAM to create a DSB within the protospacer.

The term “cancer-specific nucleic acid binding RNA” is used in accordance with its plain and ordinary meaning in light of the present disclosure and refers to a polynucleotide sequence including the crRNA sequence and optionally the tracrRNA sequence. The crRNA sequence includes a guide sequence (i.e., “guide” or “spacer”) and a tracr mate sequence (i.e., direct repeat(s)). The term “guide sequence” refers to the sequence that specifies the target site (e.g., extrachromosomal cancer-specific nucleic acid).

In general, a guide sequence is any polynucleotide sequence having sufficient complementarity with a target polynucleotide sequence to hybridize with the target sequence (i.e., an extrachromosomal cancer-specific nucleic acid) and direct sequence-specific binding of a CRISPR complex to the target sequence (i.e., the extrachromosomal cancer-specific nucleic acid). In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Sequence alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any extrachromosomal cancer-specific nucleic acid. A guide sequence is designed to have complementarity with an extrachromosomal cancer-specific nucleic acid. Hybridization between the extrachromosomal cancer-specific nucleic acid and the guide sequence promotes the formation of a CRISPR complex. Full complementarity is not necessarily required, provided there is sufficient complementarity to cause hybridization and promote formation of a CRISPR complex. A guide sequence (spacer) may comprise any polynucleotide, such as DNA or RNA polynucleotides.

In general, a tracr mate sequence includes any sequence that has sufficient complementarity with a tracr sequence (i.e., a tracrRNA sequence) to promote one or more of: (1) excision of a guide sequence flanked by tracr mate sequences in a cell containing the corresponding tracr sequence; and (2) formation of a CRISPR complex at an extrachromosomal cancer-specific nucleic acid, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin.

Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence (e.g., the extrachromosomal cancer-specific nucleic acid), it is believed that complete complementarity is not needed, provided there is sufficient to be functional. In some embodiments, the tracr sequence has at least 50%, 60%, 70%, 80%, 90%, 95%, or 99% of sequence complementarity along the length of the tracr mate sequence when optimally aligned. Where the tracrRNA sequence is less than 100 (99 or less) nucleotides in length the sequence is one of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length.

### II. Methods of Treatment

In an aspect, there is provided herein a method for treating cancer in a subject in need thereof, including administering to the subject a therapeutically effective amount of an anticancer agent, where the subject has an amplified Preiss Handler pathway gene.

In embodiments, the methods include selecting a subject having cancer, prior to administering an anticancer agent. In embodiments, methods of selecting a subject having cancer include choosing a subject that has tested positive for cancer by any diagnostic method known in the art. In embodiments, the methods include selecting a subject having a Preiss Handler pathway-dependent cancer, prior to administering an anticancer agent. In embodiments, methods of selecting a subject having a Preiss Handler pathway-dependent cancer include choosing a subject that has tested positive for cancer by any diagnostic method known in the art and where the cancer has an amplified Preiss Handler pathway gene, or the cancer is demonstrated to be dependent on the Preiss Handler pathway.

In embodiments, selecting a subject having cancer includes detecting NAPRT gene expression or NADSYN1 gene expression in the subject. In embodiments, detecting gene expression includes measuring, in a biological sample from the subject, a DNA copy number of a gene, DNA amplification of a gene, or protein expression level of a gene selected from NAPRT and NADSYN1. In embodiments, a DNA copy number of about 4 or more in any one of NAPRT and NADSYN1 indicates the subject having cancer has an amplified Preiss Handler pathway gene.

In embodiments, the cancer is selected from a Preiss Handler pathway-dependent cancer. In embodiments, the Preiss Handler pathway-dependent cancer is selected from head and neck cancer, ovarian cancer, prostate cancer, esophageal cancer, salivary gland cancer, breast cancer, liver cancer, pancreatic cancer, stomach cancer, lung cancer, bladder cancer, colon cancer, and uterine cancer. In embodiments, the cancer is head and neck cancer. In embodiments, the cancer is ovarian cancer. In embodiments, the cancer is prostate cancer. In embodiments, the cancer is esophageal cancer. In embodiments, the cancer is salivary gland cancer. In embodiments, the cancer is breast cancer. In embodiments, the cancer is liver cancer. In embodiments, the cancer is pancreatic cancer. In embodiments, the cancer is stomach cancer. In embodiments, the cancer is lung cancer. In embodiments, the cancer is bladder cancer. In embodiments, the cancer is colon cancer. In embodiments, the cancer is uterine cancer.

In embodiments, the amplified Preiss Handler pathway gene is an amplified NAPRT gene and/or an amplified NADSYN1 gene as assessed at the DNA, RNA, or protein level. In embodiments, the amplified Preiss Handler pathway gene is an amplified NAPRT gene. In embodiments, the amplified Preiss Handler pathway gene is an amplified NADSYN1 gene.

In embodiments, the anticancer agent is an antibody, an antisense nucleic acid, a gene editing reagent, a gene therapy reagent, or a small molecule. In embodiments, the anticancer agent is an antibody. In embodiments, the anticancer agent is an antisense nucleic acid. In embodiments, the anticancer agent is a gene editing reagent. In embodiments, the anticancer agent is a small molecule.

In embodiments, the anticancer agent is a NAPRT inhibitor or a NADSYN1 inhibitor. In embodiments, the anticancer agent is a NAPRT inhibitor. In embodiments, the NAPRT inhibitor is 2-hydroxynicotinic acid. In embodiments, the anticancer agent is a NADSYN1 inhibitor. In embodiments, the NADSYN1 inhibitor is (N-(3,4-dichlorophenyl)-4-{[(4-nitrophenyl)carbamoyl]amino}benzenesulfonamide). In embodiments, the NAPRT inhibitor or NADSYN1 inhibitor is an antibody, an antisense nucleic acid, a gene editing reagent, a gene therapy reagent, or a small molecule that inhibits the expression or activity of NAPRT or NADSYN1.

In an embodiment, the method of treating cancer comprises selecting a subject identified as having a Preiss Handler pathway-dependent cancer, wherein the cancer has a NAPRT gene copy number of 4 or more, and administering to the subject a therapeutically effective amount of a NAPRT inhibitor.

In an embodiment, the method of treating cancer comprises selecting a subject identified as having a Preiss Handler pathway-dependent cancer, wherein the cancer has a NADSYN1 gene copy number of 4 or more, and administering to the subject a therapeutically effective amount of a NADSYN1 inhibitor.

In another aspect, provided herein is a method for treating cancer in a subject in need thereof, including administering to the subject a therapeutically effective amount of an anticancer agent, where the subject has a NAD salvage pathway-dependent cancer.

In embodiments, the methods include selecting a subject having cancer, prior to administering an anticancer agent. In embodiments, method of selecting a subject having cancer includes choosing a subject that has tested positive for cancer by any diagnostic method known in the art. In embodiments, provided herein is a method for treating cancer in a subject in need thereof, including administering to the subject a therapeutically effective amount of an anticancer agent, where the subject has a NAD salvage pathway-dependent cancer. In embodiments, the method includes selecting a subject identified as having a NAD salvage pathway-dependent cancer.

In embodiments, the cancer is a NAD salvage pathway-dependent cancer. In embodiments, the cancer is selected from muscle cancer, brain cancer, lymph node cancer, thyroid cancer, kidney cancer, and adrenal gland cancer. In embodiments, the cancer is muscle cancer. In embodiments, the cancer is brain cancer. In embodiments, the cancer is lymph node cancer. In embodiments, the cancer is thyroid cancer. In embodiments, the cancer is kidney cancer. In embodiments, the cancer is adrenal gland cancer.

In embodiments, selecting a subject having cancer includes detecting NAPRT gene expression or protein expression or NADSYN1 gene expression or protein expression in the subject. In embodiments, detecting gene expression includes measuring in a biological sample from the subject, a DNA copy number of a gene selected from NAPRT and NADSYN1. In embodiments, a DNA copy number of 0-1 in any one of NAPRT and NADSYN1 indicates the subject having cancer has a NAD salvage pathway-dependent cancer.

In embodiments, the NAD salvage pathway-dependent cancer is not amplified in a Preiss Handler pathway gene. In embodiments, the NAD salvage pathway-dependent cancer is not amplified in a NAPRT gene. In embodiments, the NAD salvage pathway-dependent cancer is not amplified in the NADSYN1 gene.

In embodiments, the anticancer agent is an antibody, an antisense nucleic acid, a gene editing reagent, a gene therapy reagent, or a small molecule. In embodiments, the anticancer agent is an antibody. In embodiments, the anticancer agent is an antisense nucleic acid. In embodiments, the anticancer agent is a gene editing reagent. In embodiments, the anticancer agent is a small molecule.

In embodiments, the anticancer agent is a NAMPT inhibitor or a NMRK1 inhibitor. In embodiments, the anticancer agent is a NAMPT inhibitor. In embodiments, the NAMPT inhibitor is FK866 (N-[4-(1-benzoyl-4-piperidinyl)butyl]-3-(3-pyridinyl)-2E-propenamide). In embodiments, the anticancer agent is a NAMPT enzyme pathway modulator. In embodiments, the modulator is a NAMPT enhancer region inhibitor. In embodiments, the NAMPT inhibitor is administered at a low therapeutically effective amount.

In embodiments, the method includes administering both a NAMPT inhibitor and a NMRK1 inhibitor. In embodiments, the method includes administering a NAMPT inhibitor and a NMRK1 inhibitor simultaneously. In embodiments, the method includes administering a NAMPT inhibitor and a NMRK1 inhibitor sequentially. In embodiments, the method includes first administering a NAMPT inhibitor and then a NMRK1 inhibitor. In embodiments, the method includes first administering a NMRK1 inhibitor and then a NAMPT inhibitor

In an aspect, provided herein is a method of detecting NAPRT gene expression or NADSYN1 gene expression in a subject that has cancer, including measuring in a biological sample from the subject, a DNA copy number of a gene selected from NAPRT and NADSYN1. In embodiments, measuring DNA copy number includes quantifying the number of copies of a gene by qPCR. In embodiments, the gene is NAPRT In embodiments, the gene is NADSYN1.

In embodiments, the method further includes classifying the cancer as a Preiss Handler pathway-dependent cancer if the DNA copy number, RNA expression level, or protein expression level of a Preiss Handler pathway component is amplified above a threshold. In embodiments, a DNA copy number of about 4 or more in any one of NAPRT and NADSYN1 results in classifying the cancer as a Preiss Handler pathway-dependent cancer. In embodiments, a DNA copy number of about 3 in any one of NAPRT and NADSYN1 results in classifying the cancer as highly likely a Preiss Handler pathway-dependent cancer.

In embodiments, the threshold is a copy number of about 4 or more. In embodiments, the threshold is a copy number of about 4. In embodiments, the threshold is a copy number of about 5. In embodiments, the threshold is a copy number of about 6. In embodiments, the threshold is a copy number of about 7. In embodiments, the threshold is a copy number of about 8. In embodiments, the threshold is a copy number of about 9. In embodiments, the threshold is a copy number of about 10.

In embodiments, the threshold is a copy number of about 4 or more. In embodiments, the threshold is a copy number of about 4. In embodiments, the threshold is a copy number of about 5 or more. In embodiments, the threshold is a copy number of about 6 or more. In embodiments, the threshold is a copy number of about 7 or more. In embodiments, the threshold is a copy number of about 8 or more. In embodiments, the threshold is a copy number of about 9 or more. In embodiments, the threshold is a copy number of about 10 or more.

In embodiments, the method further includes administering to a subject classified as having a Preiss Handle pathway-dependent cancer an anticancer agent. In embodiments, the anticancer agent is an antibody, an antisense nucleic acid, a gene editing reagent, a gene therapy reagent, or a small molecule. In embodiments, the anticancer agent is an antibody. In embodiments, the anticancer agent is an antisense nucleic acid. In embodiments, the anticancer agent is a gene editing reagent. In embodiments, the anticancer agent is a small molecule.

In embodiments, the anticancer agent is a NAPRT inhibitor or a NADSYN1 inhibitor. In embodiments, the anticancer agent is a NAPRT inhibitor. In some embodiments, the NAPRT inhibitor is 2-hydroxynicotinic acid. In embodiments, the anticancer agent is a NADSYN1 inhibitor. In embodiments, the NADSYN1 inhibitor is (N-(3,4-dichlorophenyl)-4-{[(4-nitrophenyl)carbamoyl]amino}benzenesulfonamide).

In embodiments, the method further includes classifying the cancer as a NAD salvage pathway cancer if the DNA copy number of any one of NAPRT and NADSYN1 is selected from 0, 1, and 2. In embodiments, the method further includes classifying the cancer as a NAD salvage pathway cancer if the DNA copy number of any one of NAPRT and NADSYN1 is 0. In embodiments, the method further includes classifying the cancer as a NAD salvage pathway cancer if the DNA copy number of any one of NAPRT and NADSYN1 is 1. In embodiments, the method further includes classifying the cancer as a NAD salvage pathway cancer if the DNA copy number of any one of NAPRT and NADSYN1 is 2.

In embodiments, the method further includes administering to a subject classified as having a NAD salvage pathway-dependent cancer, an anticancer agent. In embodiments, the anticancer agent is an antibody, an antisense nucleic acid, a gene editing reagent, a gene therapy reagent, or a small molecule. In embodiments, the anticancer agent is an antibody. In embodiments, the anticancer agent is an antisense nucleic acid. In embodiments, the anticancer agent is a gene editing reagent. In embodiments, the anticancer agent is a small molecule.

In embodiments, the method includes administering to a subject classified as having a NAD salvage pathway-dependent cancer an anticancer agent where the anticancer agent is a NAMPT inhibitor or a NMRK1 inhibitor. In embodiments, the anticancer agent is a NAMPT inhibitor. In embodiments, the NAMPT inhibitor is FK866 (N-[4-(1-benzoyl-4-piperidinyl)butyl]-3-(3-pyridinyl)-2E-propenamide). In embodiments, the anticancer agent is a NAMPT enzyme pathway modulator. In embodiments, the modulator is a NAMPT enhancer region inhibitor. In embodiments, the NAMPT inhibitor is administered at a low therapeutically effective amount.

In embodiments, the method includes administering both a NAMPT inhibitor and a NMRK1 inhibitor. In embodiments, the method includes administering a NAMPT inhibitor and a NMRK1 inhibitor simultaneously. In embodiments, the method includes administering a NAMPT inhibitor and a NMRK1 inhibitor sequentially. In embodiments, the method includes first administering a NAMPT inhibitor and then a NMRK1 inhibitor. In embodiments, the method includes first administering a NMRK1 inhibitor and then a NAMPT inhibitor.

In an embodiment, the method of treating cancer comprises selecting a subject identified as having a NAD salvage pathway cancer, wherein the cancer has a NAPRT gene copy number of 0, 1, or 2, and administering to the subject a therapeutically effective amount of a NAMPT inhibitor. In an embodiment, the method of treating cancer comprises selecting a subject identified as having a NAD salvage pathway cancer, wherein the cancer has a NAPRT gene copy number of 0, 1, or 2, and administering to the subject a therapeutically effective amount of FK866 (N-[4-(1-benzoyl-4-piperidinyl)butyl]-3-(3-pyridinyl)-2E-propenamide). In an embodiment, the method of treating cancer comprises selecting a subject identified as having a NAD salvage pathway cancer, wherein the cancer has a NAPRT gene copy number of 0, 1, or 2, and administering to the subject a therapeutically effective amount of a NMRK1 inhibitor.

In an embodiment, the method of treating cancer comprises selecting a subject identified as having a NAD salvage pathway cancer, wherein the cancer has a NADSYN1 gene copy number of 0, 1, or 2, and administering to the subject a therapeutically effective amount of a NAMPT inhibitor. In an embodiment, the method of treating cancer comprises selecting a subject identified as having a NAD salvage pathway cancer, wherein the cancer has a NADSYN1 gene copy number of 0, 1, or 2, and administering to the subject a therapeutically effective amount of FK866 (N-[4-(1-benzoyl-4-piperidinyl)butyl]-3-(3-pyridinyl)-2E-propenamide). In an embodiment, the method of treating cancer comprises selecting a subject identified as having a NAD salvage pathway cancer, wherein the cancer has a NADSYN1 gene copy number of 0, 1, or 2, and administering to the subject a therapeutically effective amount of a NMRK1 inhibitor.

In embodiments, provided herein are methods for treating cancer in a subject, the method includes administering to a subject having a NAD-pathway dependent cancer a therapeutically effective amount of an anticancer agent, wherein the NAD-pathway is the Preiss Handler pathway or the NAD salvage pathway.

In embodiments, provided herein are method for detecting a NAD-pathway dependent cancer comprising measuring in a cancer sample from a subject a DNA copy number of NAPRT and/or NADSYN1 and comparing the copy number to a threshold, wherein a copy number of 4 or more indicates a Preiss Handler pathway dependent cancer, and a copy number of 0, 1, and 2 indicates a NAD salvage pathway cancer.

### III. Compositions

In an aspect, provided herein are compositions including a NAMPT inhibitor and a NMRK1 inhibitor.

In embodiments, the compositions provided herein include an NAMPT inhibitor where the NAMPT inhibitor is an antibody, an antisense nucleic acid, a gene editing reagent, or a small molecule. In embodiments, the compositions provided herein include an NAMPT inhibitor where the NAMPT inhibitor is an antibody. In embodiments, the compositions provided herein include an NAMPT inhibitor where the NAMPT inhibitor is an antisense nucleic acid. In embodiments, the compositions provided herein include an NAMPT inhibitor where the NAMPT inhibitor is a gene editing reagent. In embodiments, the compositions provided herein include an NAMPT inhibitor where the NAMPT inhibitor is a small molecule.

In embodiments, the compositions provided herein include a NAMPT inhibitor, where the NAMPT inhibitor is FK866 (N-[4-(1-benzoyl-4-piperidinyl)butyl]-3-(3-pyridinyl)-2E-propenamide).

In embodiments, the compositions provided herein include a NAMPT inhibitor, where the NAMPT inhibitor is a NAMPT enzyme pathway modulator. In embodiments, the NAMPT enzyme pathway modulator is a NAMPT enhancer region inhibitor.

In embodiments, the compositions provided herein include a NMRK1 inhibitor, where the NMRK1 inhibitor is an antibody, an antisense nucleic acid, a gene editing reagent, or a small molecule. In embodiments, the compositions provided herein include a NMRK1 inhibitor, where the NMRK1 inhibitor is an antibody. In embodiments, the compositions provided herein include a NMRK1 inhibitor, where the NMRK1 inhibitor is an antisense nucleic acid. In embodiments, the compositions provided herein include a NMRK1 inhibitor, where the NMRK1 inhibitor is a gene editing reagent. In embodiments, the compositions provided herein include a NMRK1 inhibitor, where the NMRK1 inhibitor is a small molecule.

In embodiments, the compositions provided herein include a NMRK1 inhibitor, where the NMRK1 inhibitor is a NMRK1 enzyme pathway modulator.

In embodiments, provided herein are compositions including a NAMPT inhibitor and a NMRK1 inhibitor and further including a PARP inhibitor. In embodiments, compositions provided herein include a PARP inhibitor selected from Olaparib, Rucaparib, Niraparib, Talazoparib, Veliparib, and Pamiparib. In embodiments, provided herein are compositions including a NAMPT inhibitor, a NMRK1 inhibitor and Olaparib. In embodiments, provided herein are compositions including a NAMPT inhibitor, a NMRK1 inhibitor and Rucaparib. In embodiments, provided herein are compositions including a NAMPT inhibitor, a NMRK1 inhibitor and Niraparib. In embodiments, provided herein are compositions including a NAMPT inhibitor, a NMRK1 inhibitor and Talazoparib. In embodiments, provided herein are compositions including a NAMPT inhibitor, a NMRK1 inhibitor and Veliparib. In embodiments, provided herein are compositions including a NAMPT inhibitor, a NMRK1 inhibitor and Pamiparib.

In embodiments, provided herein is a pharmaceutical composition including any one of the various compositions of the various compositions described herein and a pharmaceutically acceptable excipient.

### EXAMPLES

### Example 1: Tissue-Lineage Dependent, PH-Pathway Addition in Cancer is Driven by Gene Amplification

The experiments herein show that tissue context was the major determinant of dependence on the nicotinamide adenine dinucleotide (NAD) metabolic pathway in cancer. By analyzing more than 7,000 tumors and 2,600 matched normal samples of 19 tissue types, coupled with mathematical modeling and extensive in vitro and in vivo analyses, a simple and actionable set of ‘rules’ was identified. Data showed that if the rate-limiting enzyme of de novo NAD synthesis, NAPRT, is highly expressed in a normal tissue type, cancers that arise from that tissue will have a high frequency of NAPRT gene amplification and be completely and irreversibly dependent on NAPRT enzyme for survival. By contrast, tumors that arise from normal tissues that do not express NAPRT gene highly are entirely dependent on the NAD salvage pathway for survival. A previously unknown enhancer that underlies this dependence was identified by experiments herein. Amplification of the NAPRT gene was shown to generate a pharmacologically actionable tumor cell dependence for survival. Dependence on another rate-limiting enzyme of the NAD synthesis pathway, NAMPT, was subject to resistance by NMRK1-dependent synthesis of NAD as a result of enhancer remodeling. These results identified a central role for tissue context in determining the choice of NAD biosynthetic pathway, explained the failure of NAMPT inhibitors in past treatment regimens, and paves the way for more effective treatments.

Analysis of 63,865 samples from 216 cancer studies revealed that the rate-limiting enzymes of the Preiss Handler, salvage, and de novo NAD synthesis pathways—nicotinate phosphoribosyltransferase (NAPRT), nicotinamide phosphoribosyltransferase (NAMPT) and quinolinate phosphoribosyltransferase (QAPRT), respectively—were mutated in less than 1% of tumors. By contrast, the DNA copy numbers of NAPRT and NADSYN1 genes were increased in many cancer types, including prostate, ovarian and pancreatic cancers, in 28 out of 54 cell lines profiled from the NCI-60 panel; heatmap illustrating copy number (CN) alterations (z score) for NAMPT, NAPRT, and NADSYN1 across cancer cell types (n=54 cell types and representative FISH images of cells in metaphase from two independent experiments with similar observations displaying NAPRT and NADSYN1 gene amplification on homogenously staining regions in PH-amplified (OV4PH-amp and KYSE510PH-amp) and non-PH-amplified (H460non-PH-amp) cancer cell lines support the conclusions and in 295 out of 947 (31%) CCLE cell lines, significantly increasing gene expression.

Amplification of PH-pathway genes (NAPRT and/or NADSYN1) in 7,328 tumors of various histological types was significantly correlated with NAPRT gene expression in 2,644 matched normal tissues from which these tumors arose. Tissue-of origin NAPRT gene expression was bimodally distributed (P<0.02, Methods), and 1,475 out of 1,573 NAPRT amplified tumors (93%) arose from tissues that expressed high levels of the NAPRT transcript (P<0.0001, Methods), suggesting a role for tissue context in determining which cancers amplify NAPRT.

Data demonstrated that tissue lineage-dependent, PH-pathway addiction in cancer is driven by gene amplification. NAD+ biosynthesis pathways and gene amplification frequencies in cancer were ascertained (adeno, adenocarcinoma; CS, carcinosarcoma; LGG, low grade glioma; MBL, monoclonal B-cell lymphocytosis; NA, nicotinic acid; NEPC, neuroendocrine prostate cancer; NM, nicotinamide; NR, nicotinamide riboside; NMNAT, nicotinamide mononucleotide adenylyltransferase; QA, quinolinic acid; QAPRT, quinolinic acid phosphoribosyltransferase; squ, squamous). The data showed that tissue context determines the metabolic choice made by cancer cells (NTC: Non-Targeting Control). Unlike normal cells, PH-pathway amplified tumor cells are dependent on NAPRT for survival. P values were determined using Hartigan's dip test followed by Bayesian probability statistics and two-sided Fishers exact test or one way analysis of variance (ANOVA) with Tukey's multiple comparisons test.

Additional experimental data showed tissue lineage-dependent, PH-pathway gene amplification in cancer. NAPRT or NADSYN1 mRNA expression was plotted against putative copy number alterations from several tumor types from CCLE (n=947 from biologically independent samples including shallow/deep deletion) were produced. NAPRT or NADSYN1 mRNA expression was stratified by NAPRT and NADSYN1 copy number alterations in multiple tumor types from cBioportal (ovarian adenocarcinoma: n=403; oesophageal carcinoma: n=150; hepatocellular carcinoma: n=341; metastatic prostate adenocarcinoma: n=99; breast carcinoma: n=311; lung squamous cell carcinoma: n=163; head and neck adenocarcinoma: n=503, from biologically independent samples) were produced. Violin plots displayed median, first and third quartiles. Box and whisker plots depicting normalized NAPRT transcript level (RPKM) in 19 distinct normal tissue of origin obtained from the GTEx and TCGA portal (www.gtexportal.org; www.portal.gdc.cancer.gov/repository) were produced. Box plot with all points plotted according to the Tukey method was produced. A bar graph depicting bimodal distribution based on dip test of unimodality of two distributions stratified as ‘high’ or ‘low’ (n=2,644 biologically independent samples). To classify tissues as having ‘high’ or ‘low’ gene expression, the critical point of distribution was chosen at 10 RPKM, at which the two distributions have identical density. Data showed the Pearson correlation between expression of the NAPRT transcript (RPKM, z score) in 19 normal tissues and NAPRT or NADSYN1 copy number in 23 cancer types (n=2,644 biologically independent samples). Statistical significance for mRNA expression against putative copy number alterations was assessed using two-tailed unpaired Student's t-test.

Non-cancerous cells were able to use any of the NAD biosynthetic pathways to maintain intracellular NAD levels and did not die in response to a specific NAMPT inhibitor, FK-866, or small interfering RNA (siRNA)-mediated genetic depletion of the rate-limiting enzymes of de novo NAD synthesis, or PH or salvage pathways. Intracellular measurement of NAD+ levels were taken in non-cancer cells after treatment with increasing doses of the NAMPT inhibitor FK-866 for 72 hours. Microscopy images of non-cancer cells from one of two independent experiments, treated with increasing doses of FK-866 for 72 hours were taken. Both biological replicates showed similar results. Original magnification, ×10. Experiments were conducted related to non-cancer cells transfected with siRNAs targeting NAMPT (siNAMPT), NMRK1 (siNMRK1) and NAPRT (siNAPRT), either individually or in combination. A non-targeting control siRNA (siNTC) was used a negative control. Percentage of cell death was assessed by trypan blue exclusion assay in non-cancer cells. Experiments were conducted showing the intracellular measurement of NAD+ levels in non-cancer cells. Representative microscopy images of non-cancer cells from one of two independent experiments, transfected with siRNAs targeting NAMPT, NMRK1 and NAPRT, either individually or in combination were produced. Both biological replicates showed similar results. Original magnification, ×10. Immunoblots for cleaved caspase-3 (Cl-cas3) as a measure of cell death and to test for abundance of NAMPT, NMRK1 and NAPRT protein expression were produced. Experiments were conducted showing intracellular measurement of NAD+ levels in non-cancer cells supplemented with exogenous NAD+ (200 μM) or with the indicated precursors, nicotinic acid (NA), nicotinamide (NM) or nicotinamide riboside (NR) at a concentration of 500 μM. Data representative of five biological replicates from five independent experiments were evaluated. Data are mean±s.d. P values determined by one-way ANOVA with Tukey's multiple comparisons test.

By contrast, 29 out of 29 cancer cell lines with NAPRT amplification and/or NADSYN1 amplification (PH-amplified), but 0 out of 25 non-PH-amplified cell lines, depended on NAPRT and NADSYN1 enzymes for survival (See Table 1).

TABLE 1
Overview of 54 distinct established cancer models
of 13 histological types analyzed herein.

| Cancer cell | | |
|---|---|---|
| types applied | PH | non-PH |
| in study | amp | amp |
| H&N | 2 | 0 |
| Esophagus | 2 | 0 |
| Pancreas | 2 | 0 |
| Ovarian | 5 | 0 |
| Prostate | 3 | 0 |
| Lung | 6 | 2 |
| Colon | 5 | 2 |
| Breast | 3 | 2 |
| Brain | 0 | 9 |
| Renal | 0 | 2 |
| Melanoma | 1 | 5 |
| Leukemia | 0 | 2 |
| Cervical | 0 | 1 |
| Total | 29 | 25 |

As summarized above, tissue context determines the NAD metabolic pathway dependence of cancer cells. Both non-cancer and cancer cells (n=59 cell types) were transfected with four different siRNAs targeting NADSYN1, and a non-targeting siNTC was used a negative control. Cell death was measured by propidium iodide staining (z score). Different cancer cell types (n=54 cell lines) were transfected with the indicated siRNAs. Intracellular measurement of NAD+/NADH (left) and NAD+ levels in non-cancer (n=5 cell types) and cancer (n=21 cell types) cell lines were taken. Intracellular measurement of NAD+/NADH (left panel) and NAD+(right panel) levels in non-cancer cell lines (n=5 cell types) and cancer cell lines amplified (PH-amp) or not amplified (non-PH-amp) for the PH-pathway enzymes NAPRT or NADSYN1 (n=21 cell types). Data are representative of independent biological replicates from three independent experiments. P values determined by one-way ANOVA with Tukey's multiple comparisons test or a two-tailed unpaired Student's t-test.

Short hairpin RNAs (shRNAs) that target key enzymes of de novo NAD synthesis, or the PH and salvage pathways confirmed that PH-amplified cancer cells were entirely dependent on the PH pathway for maintenance of NAD and cell survival. By contrast, non-PH-amplified cancer cell lines depended exclusively on NAMPT and the salvage pathway. Data showed the genetic depletion of genes encoding key enzymes of NAD biosynthesis pathways combined with metabolic addbacks identify mechanistic basis of NAD pathway addiction. Colony formation assay using crystal violet staining from one of two independent experiments were conducted. Both biological replicates showed similar results. Cells stably expressing different ishRNAs were stained with crystal violet 15-18 days after transduction and selection. Immunoblots were produced for cleaved caspase-3 as a measure of cell death and to test for protein abundance for NAPRT and NAMPT in PH-amplified and non-PH-amplified cancer cells transduced with respective ishRNAs. Actin was used as a loading control. Representative blots from one of two independent experiments. Both biological replicates showed similar results.

Heatmaps illustrating absolute colony formation units were produced. Experiments for heatmap data were repeated twice. The data demonstrate that tumor cells lacking these amplicons were entirely dependent on NAMPT for survival. The addition of inducible shRNAs (ishRNAs) that target key pathway intermediates confirmed that survival dependence is completely mediated via NAD synthesis.

Cancer cell lines (n=8 cell types) amplified for the PH-pathway enzymes NAPRT or NADSYN1 were transduced independently each with a DOX-inducible ishRNA, followed by DOX treatment after puromycin selection. Non-cancer cell lines used as controls were also transduced with the indicated DOX-inducible ishRNAs. Cells were supplemented with fresh growth media and exogenous NAD+ (200 μM) or the indicated precursors, nicotinic acid (NA), nicotinic acid mononucleotide (NAMN), nicotinamide (NM), nicotinamide mononucleotide (NMN), nicotinamide riboside (NR), TRP or quinolinic acid (QA) at a concentration of 500 μM every 2-3 days.

Histone H3 lysine 27 acetylation (H3K27ac) using chromatin immunoprecipitation followed by sequencing (ChIP-seq) [See for example Refs. 15, 16] revealed a long-range, putative NAMPT enhancer 65 kb downstream of the NAMPT transcription start site on chromosome 7 (hg19: 105,856,018-105,860,658), specifically marked by H3K27ac and/or an accessible DNase I hypersensitive (DHS) signal in salvage-dependent, but not in PH-amplified cancer or normal cell lines. This 4.641-kb cis-regulatory region demonstrated potent enhancer activity when cloned either upstream or downstream of the 1.759-kb NAMPT promoter in a reporter construct and tested in the salvage-dependent cancer cell lines, but not in PH-amplified or non-cancer control cell lines.

Experiments were conducted related to the identification of an epigenetic basis for NAMPT pathway addiction in non-PH-amplified cancers. A Genome browser snapshot illustrating H3K27ac and DHS ChIP-seq signal peaks across cancer cell lines and matched tumor tissue biopsies was produced and a putative NAMPT enhancer identified. The putative NAMPT enhancer locus cloned into an engineered luciferase reporter construct, showed activity upstream or downstream of the native NAMPT promoter. Deletion mapping narrowed the NAMPT enhancer to hg19 CHr7: 105,856,574-105,857,379 (“enhancer “B”).

Experiments were conducted showing that the NAMPT enhancer drives NAD salvage-pathway addiction in cancer. Luciferase enhancer reporter assay of the putative downstream enhancer was conducted. To test the effect of a predicted enhancer, the cis regulatory region of the NAMPT locus was cloned into pGL3 reporter constructs in the direction indicated. Enhancer activity of the 4.641-kb cis-regulatory region corresponding to the H3K27ac and DHS peak was tested using a luciferase reporter assay, when present both upstream and downstream of the luciferase gene in a construct containing the NAMPT promoter. The pGL3 reporter plasmid containing the NAMPT promoter but without the enhancer region is used as a negative control (pGL3). Luciferase reporter assay measuring the enhancer activity (NAMPT-Enh) was tested in salvage-dependent, U87^{Sal-dep} and HCT116^{Sal-dep} cancer cells. Relative luciferase units are normalized to Renilla. Data showed NAMPT transcript levels as measured by qPCR (left panels), and intracellular measurement of NAD+ levels in the indicated cells transduced with the KRABdCAS9 genetic repression system. Immunoblots were produced for abundance of cleaved caspase-3 in cells transduced with the KRAB-dCAS9 genetic repression system. Experimental data showed the transcript levels of MYC, MAX, STAT3, FOXM1, and GATA3 transcription factor genes in H460^{Sa-dep} cancer cells after siRNA-mediated depletion. Bar plots were representative of five or three independent biological replicates from independent experiments. Data are mean±s.d. P values determined by one-way or two-way ANOVA with Tukey's multiple comparisons test.

Fine-mapping of the 4.6-kb putative enhancer by stepwise 1-kb deletions or insertions identified the 1-kb enhancer ‘B’ region as responsible for NAMPT enhancer activity. As demonstrated by the data, CRISPR interference using catalytically inactive Cas9 (dCas9) fused to the Krüppel-associated box (KRAB) transcriptional repressor domain [See, for Example Ref. 17 for methods] confirmed that enhancer ‘B’: (1) controls expression of the NAMPT gene; (2) is the target of H3K27 acetylation; (3) regulates intracellular NAD levels; and (4) is required exclusively for tumor cell survival in a NAD-dependent manner in salvage-dependent cancer cells. Five different single-guide RNAs (sgRNAs) were individually fused to the dCas9-expressing construct. G1=sgRNA recognizing chromosome 7 genomic loci >20 kb away from the NAMPT ‘B’ enhancer region; g2=sgRNA recognizing 4.641-kb-long cis-regulatory region; g3 and g4=sgRNAs recognizing the NAMPT ‘B’ enhancer region. Representative blots from one of two independent experiments were produced. Both biological replicates showed similar results. Actin was used as a loading control. When quantifying NAD+ and cleaved caspase-3 abundance in H460Sal-dep, U87Sal-dep, and HCT116Sal-dep cells, exogenous NAD+ (200 μM) was added to test for the rescue of the phenotype.

Experiments were conducted related to the dissection of the NAMPT enhancer and its regulation in cancer. CRISPR interference strategy was used to identify the cis-regulatory element that controls NAMPT pathway addiction in cancer. Data showed the specific enhancer elements that regulate NAMPT transcript levels. Data showed a transcription factor motif analysis for five transcription factors that bind to the NAMPT distal enhancer. Bar plots were produced showing luciferase reporter assay and H3K27ac ChIP-qPCR to demonstrate MYC-MAX dependence of NAMPT enhancer activity. Data are representative of five biological replicates from five independent experiments, and are shown as mean±s.d. P values determined by one-way ANOVA with Tukey's multiple comparisons test.

ChIP-seq data from the Encyclopedia of DNA Elements (ENCODE) project was examined and a search was performed for transcription factor motifs, focusing on five transcription factors—c-MYC, MAX, STAT3, FOXM1, and GATA3—that bind to the NAMPT distal enhancer for further analysis. siRNA-mediated knockdown demonstrated that c-MYC and MAX specifically regulate NAMPT enhancer activity in salvage-dependent cancer cells, as measured by H3K27ac ChIP combined with quantitative PCR (ChIP-qPCR) and a luciferase reporter assay. These data identified a long-range NAMPT enhancer and reveal the epigenetic mechanism that underlies the dependence of non-PH-amplified cancer cells on the NAD salvage pathway.

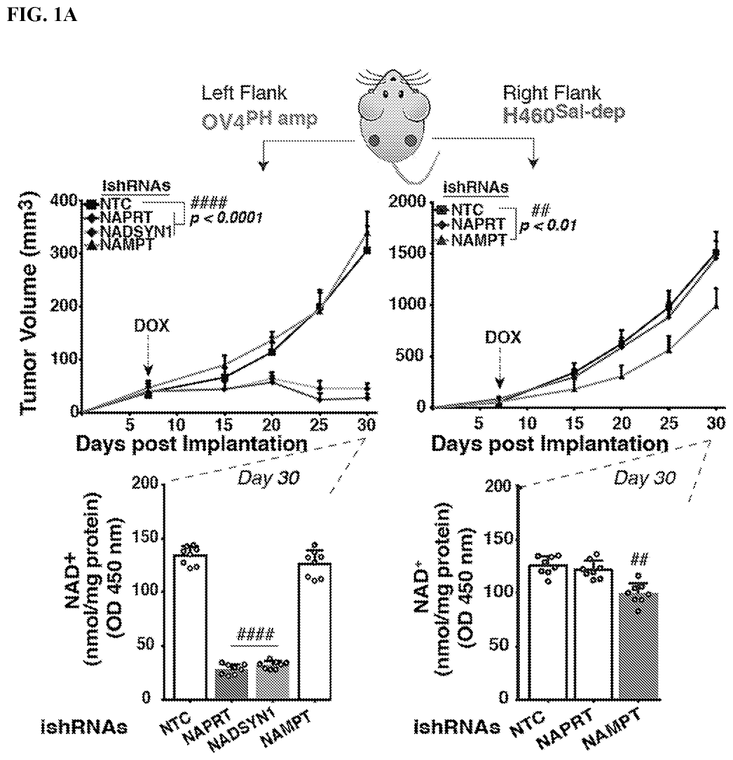

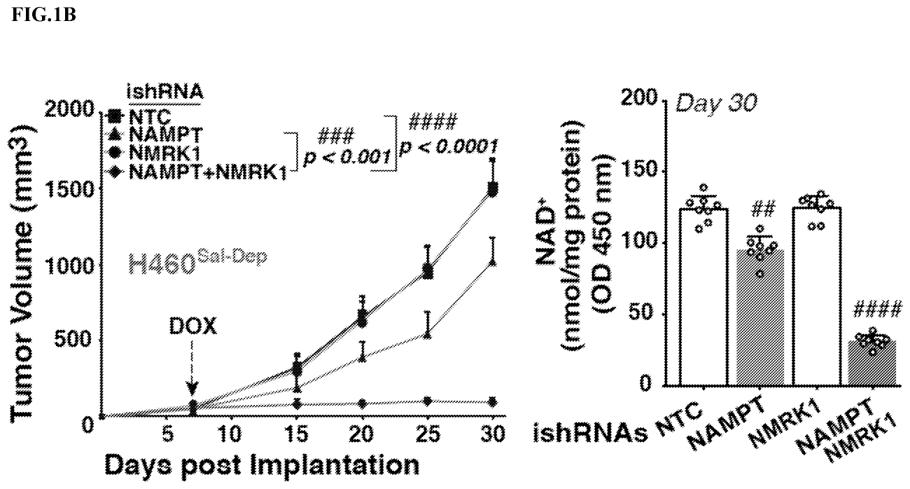

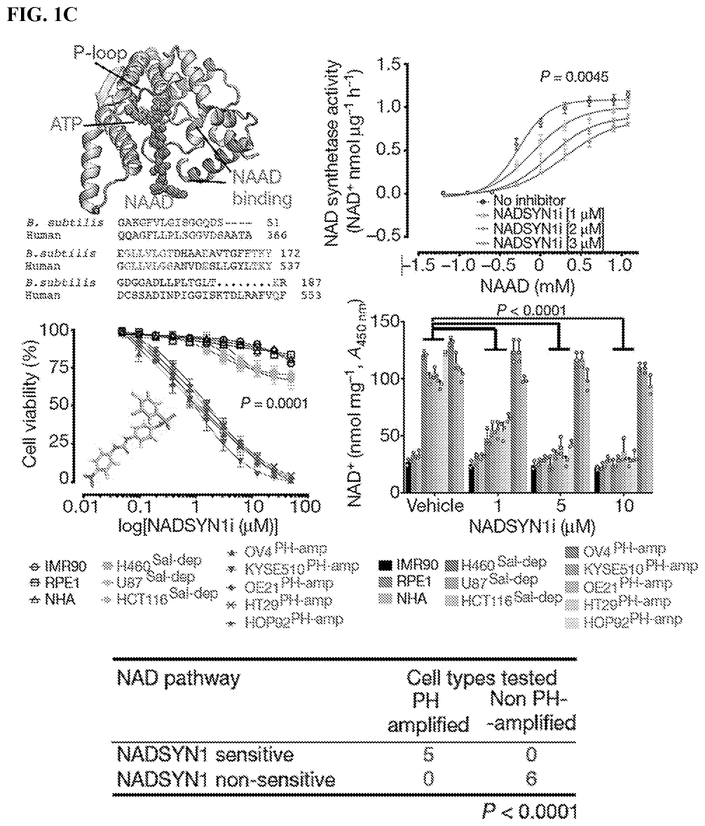

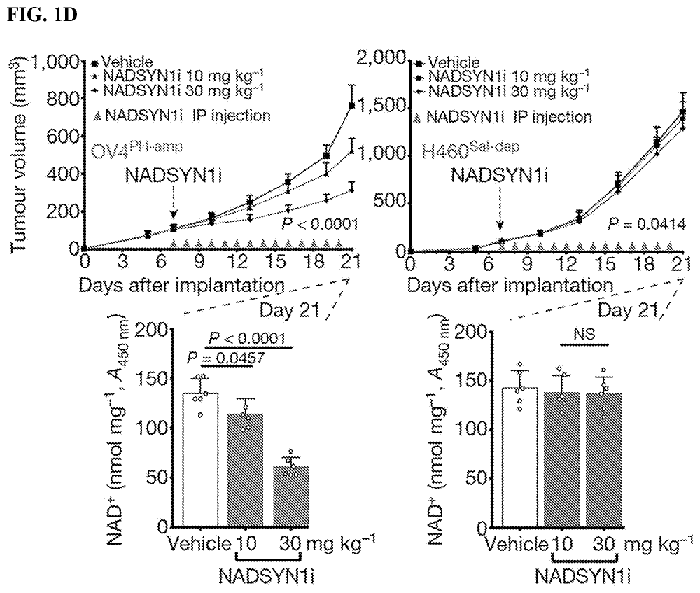

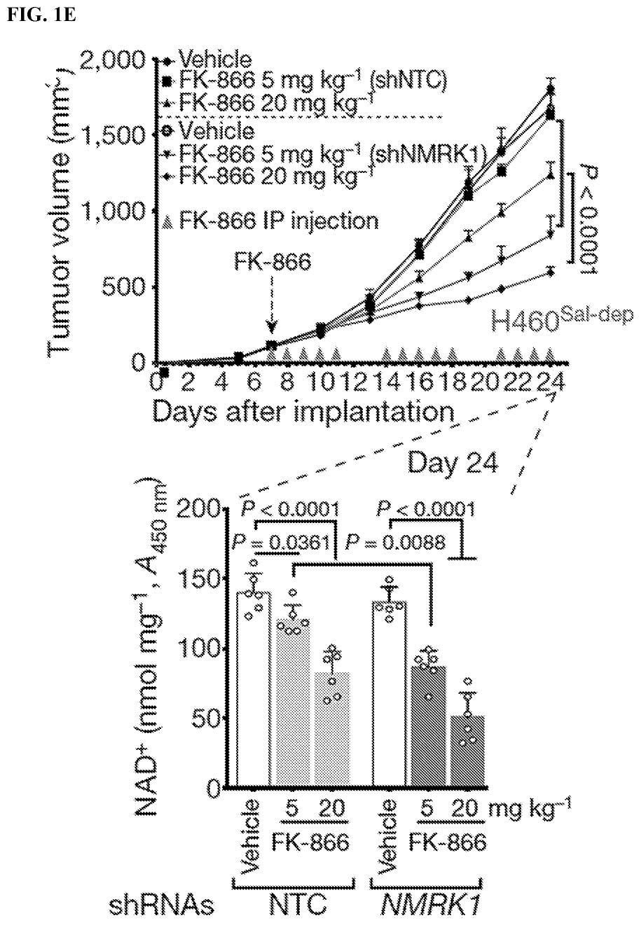

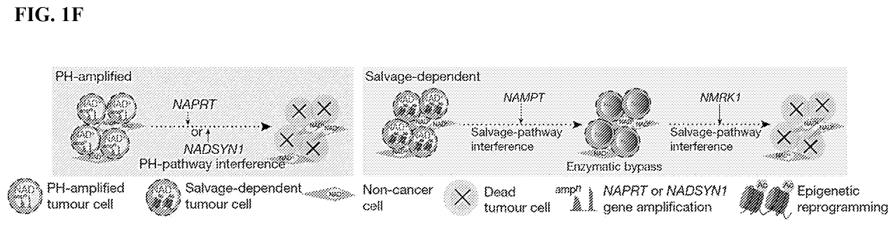

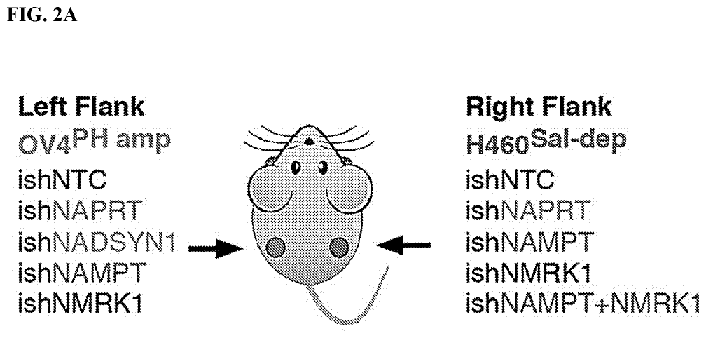

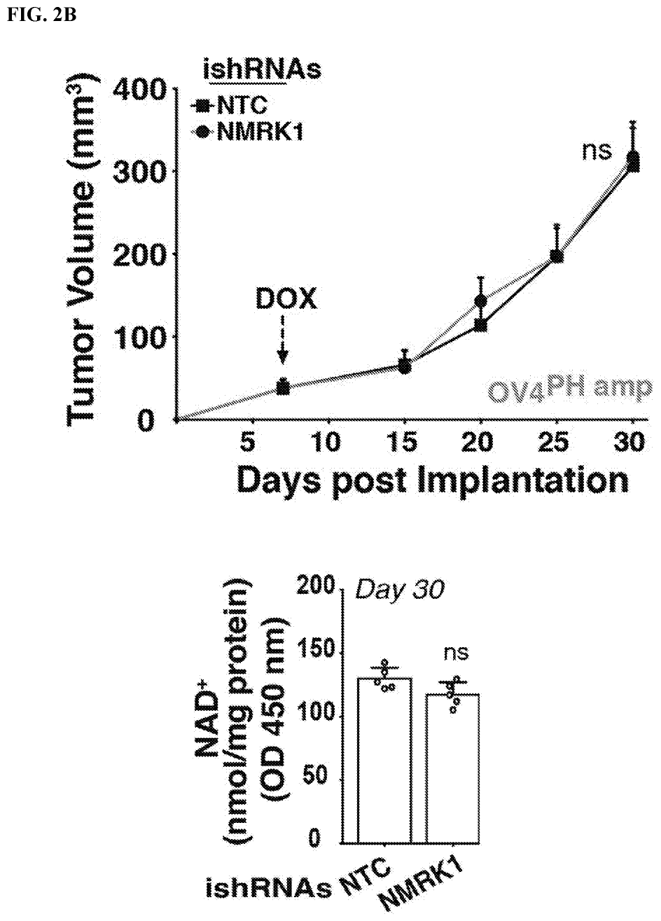

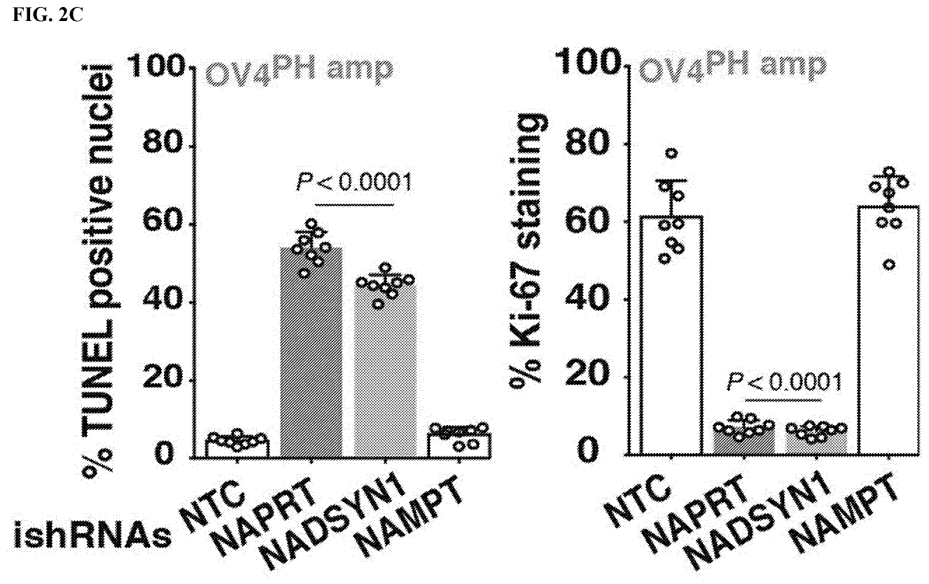

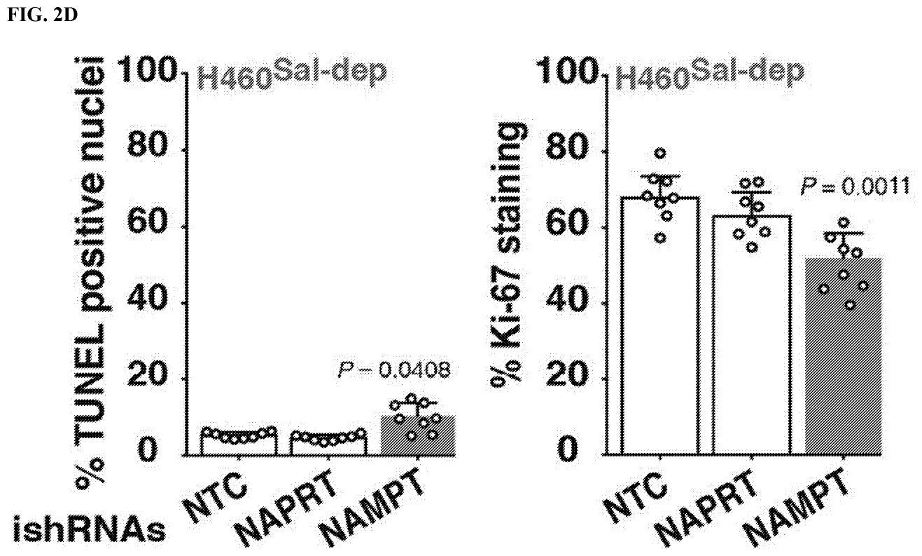

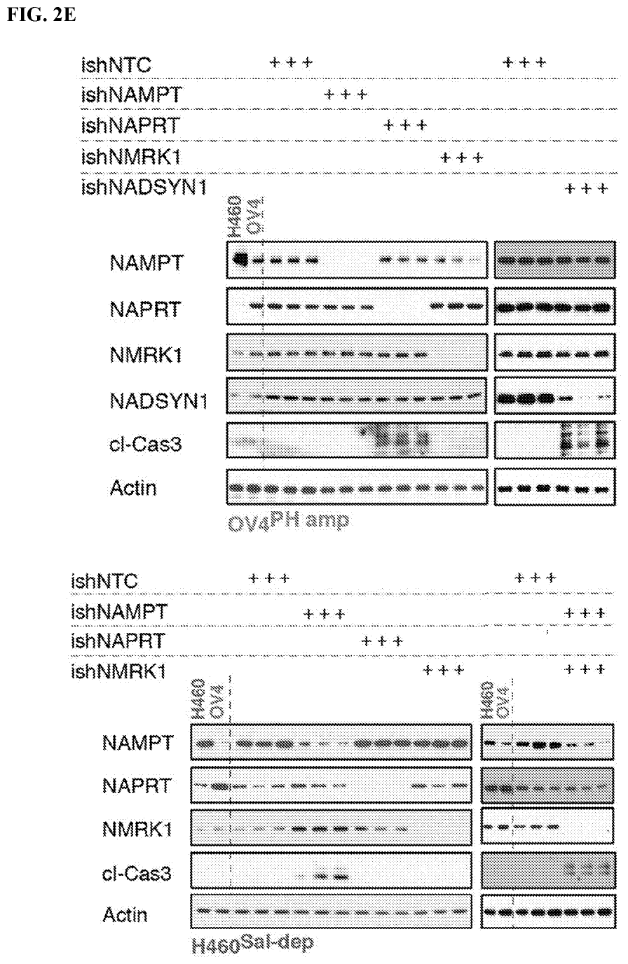

NAPRT-amplified OV4 ovarian adenocarcinoma and non-PH-pathway-amplified H460 lung cancer cells were engineered to stably express doxycycline (DOX)-inducible shRNAs that target NAPRT, NADSYN1, NAMPT, or NMRK1. The OV4 cells were implanted into the left flank, and H460 cells were implanted into the right flank of nude mice. Intratumoral levels of NAD plummeted in OV4 tumors in response to DOX-induced expression of NAPRT or NADSYN1 shRNA, causing tumors to completely and durably regress, and driving massive tumor cell death. Inducible depletion of NAMPT or NMRK1 had no effect on intratumoral levels of NAD or on the growth of OV4 tumors in vivo (FIG. 1A, FIGS. 2A-C, 2E). Therefore, NAPRT-amplified tumors use the PH pathway exclusively for NAD homeostasis and are entirely dependent on it for survival.

By contrast, H460 lung cancer cells were insensitive to depletion of NAPRT but experienced a modest reduction in intratumoral levels of NAD that was associated with a small, but significant decrement in tumor proliferation and growth in response to induced NAMPT depletion (FIG. 1A, FIGS. 2A 1D, 2E).

Experiments with in vivo genetic depletion of genes encoding key enzymes of NAD biosynthesis pathways combined with genetic rescue identified the mechanistic basis of NAD pathway addiction. OV4^{PH-amp} or H460^{Sal-dep} cells stably expressing DOX-inducible shRNA targeting the 3′ UTR of the target genes were inoculated into the left flank of individual nude mice as indicated. The same clone of the stably engineered OV4^{PH-amp} or H₄₆₀^{Sal-dep} cells but with expression of exogenous cDNA corresponding to the target not susceptible to silencing compared to the endogenous copy (ishNAPRT(+naprt−Flag)), (ishNAMPT(+nampt−Flag)) or (ishNAMPT+NMRK1(+nmrk1−Flag)) was inoculated into the right flank of individual mice as indicated. ishNTC was used as a non-targeting control inducible shRNA. Data showed tumor volume from different tumor types as indicated. Tumor volume was monitored over a 30-day period. DOX treatment was initiated on day 7 after implantation until the end of the experiment. Data showed intratumoral NAD+ measurement of nude mice bearing tumors taken at the end of experiment on day 30 for the indicated tumor types. Immuno blots were produced for NAPRT, NAMPT, NMRK1, and Flag in tumor tissues obtained from the indicated tumor types to check for protein abundance. Representative blots are from one of two independent experiments. Both biological replicates showed similar results. Actin was used as a loading control. Data are representative of eight or six independent biological replicates from two independent experiments. Data are mean tumor volume±s.e.m. (n=8 tumors per cohort), with P values determined by two-way ANOVA on repeated measurements over time. Data in scatter plots are mean±s.d., with P values determined by one-way ANOVA with Tukey's multiple comparisons test.

In skeletal muscle cells, nicotinamide riboside has been shown to be an alternative source for NAMPT-independent synthesis of NAD via the salvage pathway using NMRK kinases (See for example Ref. 18). Endogenous NMRK1 was sufficient to maintain NAD homeostasis and growth of tumor cell lines after inhibition of NAMPT.

Experimental data showed NAMPT deficiency leads to enzymatic bypass of the salvage pathway, successfully reprogramming NAD biosynthesis in cancer. Genetically engineered salvage-dependent cancer cells including H₄₆₀^{Sal-dep}, HCT116Sal-dep, and U87^{Sal-dep} were transduced with two different shRNAs against NAMPT followed by puromycin selection. A non-targeting shNTC was used a control. Data showed intracellular measurement of NAD+levels. Data showed representative images of a clonogenic survival assay using crystal violet staining from one of two independent experiments. Both biological replicates showed similar results. Data showed quantification of colony formation units. Cells were stained with crystal violet 15-18 days after seeding. Salvage-dependent cancer cells with NAMPT stably silenced grown for an extended duration of time (long-term depletion) were later silenced with shRNA against NMRK1. Immunoblots were produced for cleaved caspase-3 as a measure of cell death and to test for protein abundance of NAMPT, NMRK1, and NAPRT. Representative blots are from one of two independent experiments. Both biological replicates showed similar results. Actin was used as a loading control. Salvage-dependent cancer cells stably silenced for NAMPT and grown for an extended duration of time (long-term depletion) were later silenced for NMRK1 using siRNA. Data showed relative NMRK1 and NAMPT, NMRK2, or NAPRT transcript levels as measured by qPCR. Salvage-dependent cancer cells stably silenced for NAMPT and grown for an extended duration of time (long-term depletion), were later silenced with NMRK1 shRNA. A model was produced illustrating NAD pathway addiction in cancer is driven by two separate mechanisms. Data are representative of five, three, and two independent biological replicates from independent experiments. Data in scatter plots are mean±s.d., with P values determined by one-way ANOVA with Tukey's multiple comparisons test.

The model demonstrates tissue context-based amplifications of genes encoding key enzymes (NAPRT and NADSYN1) of the PH-pathway and subsequent tumor cell dependence that is absolute and not subjected to enzymatic bypass rewiring. By contrast, epigenetically determined dependence on the NAMPT driven salvage-pathway is subject to enzymatic bypass, requiring combination therapies. In all panels, for short-term depletion, cells were seeded 7-10 days after transduction/selection, and for long-term depletion, cells were seeded ≥30 days after transduction/selection.

Dual inhibition of both NAMPT and NMRK1 caused intratumoral levels of NAD to decrease, resulting in complete and durable tumor regression and massive tumor cell death in vivo (FIG. 1B, FIG. 2E10). These data demonstrate the need for combined inhibition of NAMPT and NMRK in non-PH-amplified tumors.

### Example 2: Inhibition of NAD Pathways

To determine whether biosynthetic NAD dependencies in cancer are pharmacologically actionable, experiments were conducted with a substrate competitive inhibitor of bacterial NADSYN1 (See for example Ref. 19), N-(3,4-dichlorophenyl)-4-{[(4-nitrophenyl)carbamoyl]amino}benzenesulfonamide, hereafter referred to as NADSYN1i. While there appears to be no available crystal structures for the human NADSYN1 enzyme, there are several high-resolution structures available for various bacterial species, such as Bacillus subtilis (Protein Data Bank (PDB) accession 1EE1) (See for example Ref 20). Sequence alignments between the human and B. subtilis NADSYN1 show limited conservation (23%), but a remarkably high degree of sequence conservation in the binding sites for nicotinic acid adenine dinucleotide (NAAD) and ATP, as depicted in (FIG. 1C) (and see for example Ref 21). NADSYN1i has been suggested to bind competitively to the NAAD-binding site of B. subtilis (see for example Refs. 19, 22, 23), suggesting that NADSYN1i could also inhibit the human NADSYN1 enzyme. Data showed that NADSYN1i inhibited purified human NADSYN1 enzyme activity in a dose-dependent manner (FIG. 1C) and potently and selectively inhibited the growth of PH-amplified cancer cell lines and reduced NAD levels while having negligible effect on normal cells of non-PH-amplified cancer cell lines (FIG. 1C). NADSYN1i significantly inhibited the growth of NAPRT-amplified OV4 ovarian tumors and reduced intratumoral levels of NAD in mice in a dose-dependent manner and had minimal effect on levels of NAD or tumor growth in salvage-dependent H460 lung tumors (FIG. 1D).

By contrast, the NAMPT inhibitor FK-866 selectively inhibited the growth of salvage-dependent cancer cell lines in vitro (heatmap data not shown; FIGS. 13A-C) and inhibited the growth of H460 lung tumors in mice in a dose-dependent fashion, but had no effect on PH-amplified OV4 tumors in vivo (FIG. 1E).

Experimental data showed genetic depletion of NMRK1 in non-PH-amplified tumor cells enhances sensitivity to FK-866 inducing tumor cell death. Data not shown provided a heatmap illustrating cell death measured by propidium iodide staining (z score). Cancer cells were treated with increasing doses of FK-866 for 72 hours. Data showed intracellular measurement of NAD+ levels. Immunoblots were produced for cleaved caspase-3, in salvage-dependent cancer cells treated with 10 nM FK-866 for 72 hours. Cells were supplemented with exogenous NAD+ (200 μM) or with the indicated precursors (NM, NMR, NR, NA) at a dose of 500 μM. Results showed cell viability of non-cancer and cancer cells (PH-amp and sal-dep) stably silenced with shRNA against NMRK1, treated with increasing doses of FK-866 for 72 hours. OV4^{PH-amp} (top) and H460^{Sal-dep} (bottom) cells stably expressing shRNA against the target gene NMRK1 were implanted subcutaneously. Data showed tumor volume of nude mice bearing stably engineered OV4^{PH-amp} cells implanted subcutaneously. Tumor volume was monitored over a 24-day period. Mice were injected intraperitoneally with FK-866 twice daily. Data showed intratumoral NAD+ measurement of nude mice bearing stably engineered OV4^{PH-amp} tumors, taken at the end of experiment on day 24. Immunoblots were produced for cleaved caspase-3 as a measure of cell death and to test for protein abundance for NMRK1 in tumor tissues obtained from the indicated tumor types. Representative blots are from one of two independent experiments. Both biological replicates showed similar results. Actin was used as a loading control. Data are representative of three, eight or six independent biological replicates from independent experiments. Data in scatter plots are mean±s.d., with P values determined by one-way ANOVA with Tukey's multiple comparisons test. For cell viability data, P values were determined by two-tailed unpaired Student's t-test. Data in mean tumor volume±s.e.m. (n=8 tumors/cohort), with P values determined by two-way ANOVA on repeated measurements over time.

Consistent with a central role for NMRK1 in bypassing NAMPT dependence, depletion of NMRK1 by shRNA knockdown significantly lowered the dose of FK-866 needed to inhibit growth of salvage-dependent, non-PH-amplified tumors in vitro and in vivo (FIG. 1E, 1F). Data not shown provided a heatmap illustrating cell death measured by propidium iodide staining (z score). Cancer cells were treated with increasing doses of FK-866 for 72 hours.

Doxycycline (DOX) treatment or drug administration was initiated on day 7 after implantation once the tumors were visible. Data are representative of eight (FIGS. 1A, 1B, 1D, 1E: tumor volumes; FIG. 1A, 1B: NAD+ levels), six (FIG. 1D, 1E: NAD+ levels), and three (FIG. 1C) biological replicates. Data in FIG. 1A (top panels), FIG. 1B (left panel), FIG. 1D (top panels), and FIG. 1E (top panel) are mean tumor volume±s.e.m. (n=8 tumors per cohort from two independent experimental repeats). All bar plots and data in FIG. 1C (top right panel, middle left panel) are mean±s.d. P values determined by two-sided Fisher's exact test (FIG. 1C), two-way ANOVA on repeated measurements over time (tumor volume data in FIGS. 1A, 1B, 1D, and 1E), or one-way ANOVA with Tukey's multiple comparisons test (bar plots in FIGS. 1A-E, and non-linear curves in FIG. 1C).

Neither NAPRT nor NAMPT overexpression sensitized non-cancer cells (IMR90 and RPE-1) to NADSYN1i or FK-866. Furthermore, neither overexpression of NAMPT in OV4 cells nor overexpression of NAPRT in H460 cancer cells enabled tumor cells to switch their pathway dependence.

Experimental data showed overexpression of rate-limiting NAD biosynthesis enzymes is not sufficient to generate or reverse metabolic addiction. Immunoblots were produced for cleaved caspase-3 as a measure of cell-death and to test for abundance of NAMPT and NAPRT protein expression in non-cancer cells (IMR90, RPE-1) stably overexpressing NAMPT or NAPRT. Protein lysates from etoposide-treated H460 cancer cells were used as a control. Data showed percentage cell death as assessed by trypan blue exclusion assay. Data showed intracellular measurement of NAD+ levels. Immunoblots were produced for cleaved caspase-3 as a measure of cell death and to test for abundance of NAPRT (in H460^{non-PH-amp}) and NAMPT (in OV4PH-amp) protein expression. Protein lysates from etoposide-treated H460 or OV4 cancer cells were used as a control. Data showed percentage cell death as assessed by trypan blue exclusion assay. Data showed intracellular measurement of NAD+ levels, after stable overexpression of NAPRT or NAMPT in H460 or in OV4 cancer cells as indicated. Stably engineered non-cancer and cancer cells after selection were treated with FK-866 (10 nM) or NADSYN11i (2 μM) as indicated for 72 h. Representative blots are from one of two independent experiments. Both biological replicates showed similar results. Actin was used as a loading control. Data are representative of five and three independent biological replicates from independent experiments. Data are mean±s.d. P values determined by two-way ANOVA with Tukey's multiple comparisons test. This demonstrates that further changes are likely to occur during tumorigenesis that contribute to NAD pathway addiction.

The results presented here suggest that dependence on the NAD pathway in cancer arises from tissue-lineage-based gene amplification and epigenetic remodeling, revealing genotype-selective, pharmacologically actionable dependencies that may be used to develop more effective, precision cancer treatments that target NAD metabolism.

### Example 3: Materials and Methods

### Cell Lines and Cell Culture

The NCI-60 cell-line panel (gift from A. Shiau, obtained from NCI) was grown in RPMI-1640 with 10% fetal bovine serum (FBS) under standard culture conditions. LNCaP, PC9, CAPAN1, U-87, HEK293, IMR90, HeLa, RPE-1, and MCF10A cells used in this study were obtained from ATCC and grown according to ATCC recommendations. Normal human astrocytes were obtained from Lonza and cultured according to Lonza-specific recommendations. GBM39 and GBM6 patient-derived neurosphere lines were cultured in NeuroCult medium supplemented with epidermal growth factor, fibroblast growth factor, and heparin. KYSE510, KYSE140, and BHY cells were obtained from Leibniz-Institut DSMZ-Deutsche Sammlung von Mikroorganismen und Zellkulturen GmbH, Germany, and OE21 cells were obtained from Sigma and maintained in RPMI-1640 with 10% FBS under standard culture conditions. Cell lines obtained from NCI, ATCC, DSMZ, and Sigma were not authenticated. All cell lines were tested for mycoplasma contamination. All cells were cultured in a humidified incubator with 5% CO₂ at 37° C. Standard cell culture media formulations for both cancer and non-cancer models were supplemented with nicotinamide (4 mg ml⁻¹) and tryptophan (TRP, 16 mg ml⁻¹) and thus far have not been shown to contain traces of nicotinic acid and nicotinamide riboside as NAD precursor. To address this issue, in the present experimental cell culture model system, cells not only had constant access to nicotinamide and TRP, but also had access to the two missing precursors, nicotinic acid (4 mg ml⁻¹) and nicotinamide riboside (4 mg ml⁻¹).

### Reagents and Chemicals

Nicotinamide (N0636), nicotinic acid (N0761), and nicotinic acid mononucleotide (NAMN, N7764), TRP (T8941) were purchased from Sigma-Aldrich. Nicotinamide mononucleotide (NMN, 1094-61-7), quinolinic acid (89-00-9), nicotinamide riboside (1341-23-7), and FK-866 (658084-64-1) were purchased from Cayman chemicals, NADSYN1i was purchased form Vitas-M Laboratory (STK459768).

### Plasmids

A luciferase reporter system was developed to assess genomic DNA fragments for promoter and enhancer activity. The NAMPT promoter, when cloned upstream of the reporter gene is expected to activate luciferase reporter gene in a plasmid that does not already contain a promoter, whereas an enhancer activates the luciferase reporter gene when cloned upstream or downstream of this reporter gene in a plasmid already containing a NAMPT promoter. The putative NAMPT enhancer (hg19_dna Chr7: 105,856,018-105,860,658) and the established NAMPT promoter (hg19_dna chr7:105,925,229-05,926,250) were PCR-amplified (Clontech, PrimeSTAR GXL DNA R050A) from human genomic DNA (bacterial artificial chromosomes, BACPAC Resources Center (BPRC), clone RP11-151E21, chromosome: chr7: 105,925,229-105,926,250). The pGL3 Basic (Promega, E1751) plasmid was digested with BglII/HindIII (New England Biolabs (NEB)) and the NAMPT promoter insert was ligated into a position upstream of the luciferase reporter gene generating pGL3-p reporter construct. To clone the putative NAMPT enhancer, pGL3-p promoter plasmid was digested with MLUI/XHOI (NEB) and the putative NAMPT enhancer insert was ligated into a position upstream of the NAMPT promoter and luciferase reporter gene24, generating the NAMPT-Enh reporter construct (clone upstream). To clone the putative NAMPT enhancer downstream of the reporter gene, pGL3-p reporter construct was digested with XBAI and the putative NAMPT enhancer insert was ligated into a position downstream of the NAMPT promoter and luciferase gene24, generating the NAMPT-Enh reporter construct (clone downstream). The internal region of the NAMPT enhancer was identical in both inserts, allowing cloning of a single PCR product into either construct. To dissect the NAMPT enhancer core, deletion mutants were generated using a step-wise site-directed mutagenesis approach (Agilent, QuikChange II Site-Directed Mutagenesis 200523) to delete small ˜1-kb enhancer fragments from the NAMPT-Enh reporter construct generating distinct NAMPT-Enh reporter constructs (BCDE; ACDE; ABDE; ABCE; ABCD). To individually clone small putative NAMPT enhancer fragments, the pGL3-p promoter plasmid was digested with MluI/XhoI, and ˜1-kb long NAMPT enhancer fragment inserts PCR-amplified from human genomic DNA. These NAMPT enhancer fragments were ligated into a position upstream of the NAMPT promoter and luciferase reporter gene, generating distinct NAMPT-Enh reporter constructs (A; B; C; D; and E). Stable cell lines overexpressing NAMPT, NAPRT, or NMRK1 were established using the lentiviral expression system. In brief, nampt-FLAG, naprt-FLAG, or nmrk1-FLAG plasmids were generated by cloning the respective cDNA into the p3×FLAG-tagged pLV cs2.0 lentiviral expression vector. The plasmid vector was digested using BAMHI and XMAI restriction sites. The PCR products were cloned into the digested plasmid using ligation independent cloning methodology using exonuclease III. Primers are listed in the Sequence Listing provided herewith.

### Pan-Cancer Copy Number Alteration Analysis

Pan-cancer copy number alteration data from 7,328 tumor-tissue samples of 23 histological types were obtained from The Cancer Genome Atlas (TCGA). Copy number alteration data for the entire cohort were downloaded and analyzed using cBioPortal [See, for example, Ref 25] (www.cbioportal.org). Copy number amplification datasets within the portal were generated by the GISTIC algorithm identifying significantly altered regions of amplification or deletion across sets of patients. GISTIC-derived copy number analysis indicate copy-number level per gene, in which ‘−2’ is a deep loss (homozygous deletion), ‘−1’ is a shallow loss (heterozygous deletion), ‘0’ is diploid, ‘1’ indicates a low-level gain, and ‘2’ is a high-level amplification. mRNA data used for this analysis were RNA Seq V2 RSEM.

### Estimation of Lineage-Specific Amplification Frequency Model

Basal-normalized NAPRT transcript expression (RPKM, z score) from 19 normal tissues of origin (2,644 normal tissue samples) was obtained from GTEx [See, for example, Ref. 26] and TCGA [See, for example, Ref 27] data to compute correlations between tissue-specific gene expression and tissue lineage-dependent PH-pathway gene amplification from matched 23 tumor types (7,328 tumor tissue samples). Processed RNA-seq files for each sample were downloaded from GDC (portal.gdc.cancer.gov/repository) and GTEx data portals (gtexportal.org/home/datasets). If the rate-limiting enzyme (NAPRT) of the de novo NAD biosynthesis PH pathway is highly expressed in a normal tissue type, cancers that arise from that tissue will have high amplification frequency of genes encoding key enzymes (NAPRT, NADSYN1) of the PH pathway. To compute tissue lineage-dependent association between genotype and NAPRT gene expression, Pearson correlation was tested between basal NAPRT transcript expression (RPKM) in the normal tissue of origin versus frequency of PH-pathway copy number amplification in the corresponding tumor tissue types. This statistically confirmed a strong correlation between normalized transcript expression of the NAPRT enzyme in a normal tissue of origin and the probability of copy number amplification of PH-pathway enzymes in corresponding tumor tissue type. To further compute the significance of this observation, three assertions were made. Specifically, if samples from a normal tissue of origin, T, have ‘high’ expression of the NAPRT gene, close to 50% of the tumor samples from T have PH-pathway copy number amplification. By contrast, if a normal tissue of origin, T, has ‘low’ expression, less than 10% of the tumor samples from T contain PH-pathway copy number amplification.

1. Basal-normalized NAPRT transcript expression (RPKM) in normal tissue of origin in samples across all normal tissue types are bimodally distributed and can be treated as being sampled from one of two distributions (denoted as ‘high’ and ‘low’) (dip test of unimodality).

2. Basal-normalized NAPRT transcript expression in all samples from a tissue can be assigned to one of the two distributions but not both. Therefore, each tissue can be classified as having ‘high’ or ‘low’ expression of the gene.

3. Frequency of copy number amplification in a tissue is strongly associated with its classification as ‘high’ or ‘low’ (two-sided Fisher's test).

For assertion 1, the distribution of normalized NAPRT gene expression (RPKM) was computed. All normalized NAPRT transcript expression data were combined from 2,644 samples from 19 normal tissues of origin available from the TCGA and GTEx project and generated a frequency distribution. Using Hartigan's dip test [See, for example, Ref. 28] with a null hypothesis of unimodality, a P value of 0.0235899 was obtained, suggesting that the null hypothesis can be rejected. The critical point of the distribution as 10 RPKM was chosen. The critical point is defined as the point at which the two distributions have identical density. Given NAPRT gene expression x from a sample, let P(x|C1) denote the probability that it is drawn from C1. Assuming normality of the two distributions based on the law of large numbers, the mean and standard deviation of each distribution to obtain C1(μ1, σ1) and C2(μ2, σ2) were computed. For the 2,644 samples, the values obtained were (μ1, σ1)=(6.1935, 1.9625), and (μ2, σ2)=(30.906, 13.8312) and refer to C1 and C2 as ‘low’ and ‘high’, respectively.

The following was set,

$$P ( x \vert {C}_{1} ) ∝ \frac{1}{\sqrt{2 {πσ}_{1}^{2}}} exp ( - \frac{{( x - {μ}_{1} )}^{2}}{{σ}_{1}^{2}} )$$

Tissue can be modeled as a collection of independent sample NAPRT gene expression values, denoted by T={x1, x2, . . . , xk}. The posterior probability of a distribution label C for tissue type T can be computed using the Bayesian formula,

$$\begin{matrix}P ( {C}_{1} \vert T ) = \frac{P ( T \vert {C}_{1} ) P ( {C}_{1} )}{P ( T \vert {C}_{1} ) P ( {C}_{1} ) + P ( T \vert {C}_{2} ) P ( {C}_{2} )} \\ = \frac{{\prod }_{x ∈ T} P ( x \vert {C}_{1} ) P ( {C}_{1} )}{\begin{matrix}{\prod }_{x ∈ T} P ( x \vert {C}_{1} ) P ( {C}_{1} ) + \\ {\prod }_{x ∈ T} P ( x \vert {C}_{2} ) P ( {C}_{2} )\end{matrix}}\end{matrix}$$

$$P ( {C}_{2} \vert T ) = 1 - P ( {C}_{1} \vert T )$$

To validate the final assertion, 7,328 tumor samples from 23 tumor types were obtained and assigned to each sample a classification of ‘high’ or ‘low’, based on tissue type, as well as a binary classification of containing a copy number amplification or not. The 2×2 contingency table for the two-sided Fisher's exact test partitions 7,328 tumor samples from 23 tumor types based on the amplification frequency of genes encoding key enzymes (NAPRT and NADSYN1) of the PH pathway and basal NAPRT transcript expression classification. The Fisher's exact test was used to test for a null hypothesis of no association and obtained a P-value of 1.7259×10⁻²¹¹ suggesting that the null hypothesis can be rejected.

Transient and Stable Knockdown of Genes Using siRNA, shRNA, or ishRNA.

Transient gene-silencing experiments were performed reverse transfecting siRNAs using Lipofectamine RNAiMAX Transfection Reagent (Thermo Fisher Scientific) in growth media supplemented with 10% FBS. Growth media was replaced 24 hours after transfection, and cells were collected 72 hours after transfection. ON-TARGETplus SMART siRNA pools and their deconvoluted versions (Dharmacon), also including Silencer select siRNA's (Ambion-Thermo Fisher Scientific) were used to target NAMPT, NMRK1, NMRK2, NAPRT, or NADSYN1. Silencer Select Negative Control 2 siRNA (Ambion-Thermo Fisher Scientific) that does not target any gene product was used as a negative targeting control. For generation of stable knockdown of cell lines, lentiviral packaging/delivery system was performed. shRNA expressing pLKO.1 vector was introduced into cancer cell lines by lentiviral infection. Recombinant lentiviral particles were generated by transient transfection of HEK293T cells following a standard protocol. HEK293T cells were plated in a 100-mm dish and transfected (Xtremegene, Roche) with 6 μg of lentiviral DNA and lentiviral packaging system consisting of 0.6 μg of Rev, 0.6 μg of Gal/pol, 0.6 μg of TAT and 6 μg of VSVG. Viral supernatant was collected at 48 hours and 72 hours post-transfection, centrifuged and filtered (0.45 m) to remove any HEK293T cells. For transduction and stable knockdown of cell lines, lentiviral particles generated were added to cell culture medium containing 4 μg ml⁻¹ polybrene (Millipore). Forty-eight hours after infection, cells were selected using 1 mg ml⁻¹ puromycin for at least one week before being used for further experiments. For generation of stable inducible knockdown of cell lines, lentiviral delivering system similar to the procedure described above was performed with the exception of using SMARTvector inducible system (Dharmacon) and also that cells were maintained in dialyzed 10% FBS when generating lentiviral packaging/delivering system for transduction. For inducible RNAi experiments, stable knockdown of respective genes was induced using doxycycline at concentrations of 1.0-2.0 mg ml⁻¹. Sequence of siRNA, shRNA, ishRNA, and relevant controls used in the study are listed in SEQ ID Nos: 1-187.

### Cell-Death, Apoptosis, Viability and Clonogenic Assay.

To examine cell death, cells were treated as indicated in the figure legends and stained with propidium iodide (Sigma-Aldrich). Adherent and floating cells were then analyzed by flow cytometry using the BD LSRII flow cytometer (BD Biosciences). Data analysis was performed using FlowJo. For the siRNA screen, cells were seeded in duplicate for each condition in six-well culture plates at 100,000-200,000 cells per well. Cell death analyses were performed 72 hours after transfection. For the shRNA screen, cells were seeded in duplicate for each condition in 60-mm dishes at 500,000 cells per dish. Cell death analyses were performed 7-10 days after infection/selection. For apoptosis assays, adherent and floating cells were lysed and homogenized with RIPA lysis buffer and immunoblotted for cleaved caspase-3 by western blotting. When measuring apoptosis after siRNA transfection, cells were obtained 72 hours after transfection. For shRNA, cells were obtained 7-10 days after infection. To examine cell viability, cells were seeded in triplicate for each condition in six-well culture plates at 150,000-200,000 cells per well. Cell viability was analyzed 72 hours after siRNA transfection or FK-866 treatment [See, for example, Ref 29]. Total and live cells in each well were quantified by Trypan blue (Gibco) assay using a TC10 automatic cell counter (Bio-Rad). For clonogenic colony-formation assays, 300-500 cells were seeded in duplicate into six-well plates. Growth media was replaced every 2 days. At the indicated time point of around 2 weeks, remaining cells were fixed with 80% methanol and stained with crystal violet solution. Images were taken using a digital imaging scanner (Bio-Rad). Colony formation was quantified by ImageJ software (NIH).

### Immunoblotting and Antibodies.

Cells were washed twice in ice-cold PBS, scraped and collected as pellets post centrifugation at 14,000 r.p.m. for 5 min. The pelleted cells were lysed with RIPA lysis buffer supplemented with protease inhibitor cocktail and phosphatase inhibitors. Lysates were collected and centrifuged at 14,000 r.p.m. for 10 min. at 4° C. Protein concentrations were determined by Bradford Assay using the Protein Assay Dye Reagent Concentrate. Equal amounts of protein extracts were separated and immunoblotted by SDS-PAGE 4-12% NuPAGE Bis-Tris Mini Gel (Thermo Fisher Scientific). Proteins were later transferred using the Trans-Blot Turbo Transfer System (Bio-Rad) onto nitrocellulose membranes according to standard protocols. Membranes were blocked at room temperature with 5% bovine serum albumin in TBS with Tween buffer and incubated overnight with corresponding primary antibodies. Membranes were later incubated at room temperature with horseradish peroxidase-conjugated secondary antibodies. The immunoreactivity was detected with SuperSignal West Pico or Femto Chemiluminescent Substrate (Thermo Fisher Scientific). The following antibodies were used: PBEF/NAMPT (Cell Signaling Technologies (CST), D1K6D 61122), NAPRT (Thermo Fisher Scientific, PA5-31880), NAD synthetase (Abcam, ab171942), NMRK1 antibody (Santa Cruz, F-8 sc-398852), NMRK1 (Abcam, anti-C9orf95 antibody EPR11190 ab169548), NMRK2, or ITGB1BP3 (Thermo Fisher Scientific, PA5-24607).

### RNA Extraction and Quantitative PCR.

Total RNA was extracted using RNeasy Mini Kit (QIAGEN). First-strand cDNA was synthesized using the SuperScript VILO cDNA synthesis kit (Thermo Fisher Scientific). Quantitative PCR with reverse transcription (qRT-PCR) was performed using the iQ SYBR Green Supermix (Bio-Rad) on the CFX96 Touch Real Time PCR Detection system (Bio-Rad) following the manufacturer's instructions. Results were normalized to TBP, B2M, YWHAZ, or RPL13A as the reference gene. Results were analyzed using the ΔCₜ. Primer sequences are listed in the Sequence listing provided herewith.

### DNA Copy Number.

Copy number analyses of NAPRT, NADSYN1, NAMPT, NMRK1, and NMRK2 were quantified by qPCR, using the CFX96 Touch Real Time PCR Detection system (Bio-Rad). RPPH1 and HBB were used as single-copy reference control. PCR primer sequences for NAPRT, NADSYN1, NAMPT, NMRK1, NMRK2, RPPH1, and HBB are listed in Sequence Listing submitted herewith. PCR primer (IDT) and the region of interest was selected by Primer Blast (www.ncbi.nlm.nih.gov/tools/primer-blast/) and USCS genome browser (genome.ucsc.edu/cgibin/hgGateway). Genomic DNA from 5 non-cancer and 54 cancer cell models was investigated. PCR reactions were carried in a final volume of 20 μl containing 20 ng genomic DNA (Qiagen), 300 nM primer and 1×SYBR green PCR Master Mix (Bio-Rad). PCR conditions were as follows: one cycle at 95° C. for 5 min, followed by 40 cycles each at 95° C. for 15 s and 60° C. for 1 min. Samples were analyzed in triplicates. Each amplification reaction was checked for the absence of non-specific PCR products by melting curve analysis. Copy number analysis of NAPRT, NADSYN1, NAMPT, NMRK1, and NMRK2 was carried out using the comparative Ct method after validating that the efficiencies of PCR reactions of reference controls and genes under investigation were equal. Increase in gene copy number by 3-4 copies was defined as ‘gain’ while increase of less than or equal to 5 copies (≥5) was categorized as ‘amplification’. Primer sequences are provided in the Sequence Listing provided herewith.

### Fluorescence In Situ Hybridization.

Cells in log-growth phase were arrested in metaphase by the addition of KaryoMAX (Gibco) for 2 h. Cells were then collected and resuspended in a hypotonic solution (0.075 M KCl) for 15-30 min. Carnoy's fixative (3:1 methanol:glacial acetic acid) was added to stop the reaction. Double FISH was performed on the fixed metaphase spreads by adding the appropriate target DNA and centromere FISH probe. DNA was denatured at 75° C. for 3-5 min. and the slides were allowed to hybridize overnight at 37° C. in a humidified chamber. Post overnight incubation slides were washed in pre-warmed 0.4×SSC at 50° C. for 2 min, followed by a final wash in 2×SSC 0.05% Tween-20. DAPI was used to counterstain metaphase cells, and interphase nuclei and images were captured [See, for example, Ref. 30]. FISH probes were purchased from Empire Genomics.

### Nad Measurement.

Intracellular NAD pools were measured using NAD/NADH kit from Abcam (ab65348) according to the manufacturer's instructions.

### NADSYN1 Enzyme Activity Assay.

NADSYN1 activity of the human recombinant protein was based on fluorometric measurements of NAD⁺ formed in the absence and presence of NADSYN1i. Purified recombinant NADSYN1 (0.4 μg) was incubated with varying concentrations of NAAD+ (0-12 mM; 1 mM for a standard reaction) in the presence of saturating glutamine (20 mM) or 20 mM NH4Cl as indicated in the reaction mixture (50 mM HEPES (pH 8.8), 2 mM ATP, 56 mM KCl, 5 mM MgCl2, and 10 μg of bovine serum albumin. When glutamine was used as a substrate to measure NADSYN1 activity, 50 mM Tris-HCl (pH 7.5) was included instead of 50 mM HEPES (pH 8.8). The reaction mixture was incubated for 30 min at 37° C. The enzyme reaction was terminated by adding 7 N NaOH and then incubated at 37° C. for 30 min to obtain the fluorescent product. Final product of the assay was measured by detecting Fluorescence using 380 nm for excitation and 460 nm for emission. Fluorescence intensity of NAD⁺ standards at known concentrations under similar reaction conditions was used to calculate the amount of NAD⁺. Specific NADSYN1 activity was determined by subtracting the NAD⁺ content of enzyme-deficient blanks from the NAD⁺ content of the complete reaction mixture [See, for example, Refs. 21, 31, 32].

### Metabolic Rescue Experiments.

For NAD⁺ rescue experiments, 200 μM of NAD⁺ (Sigma-Aldrich) was added to growth medium. For metabolic precursor rescue experiments, 500 μM of nicotinic acid, NAMN, nicotinamide, NMN, nicotinamide riboside, TRP, or quinolinic acid was added independently to growth medium. Cells were treated with NAD⁺ or with individual precursors for the time periods as indicated in figure legends.

### ChIP-Seq Analysis.

ChIP-seq data for H3K27ac, DHS, and transcription factor CHiP-seq data for c-MYC, MAX, STAT3, FOXM1, and GATA3 are public datasets available from the ENCODE consortium, UCSC. The Fisher's exact test was performed to test the null hypothesis of no association and obtained a P value of 4.1×10⁻⁵, suggesting that the null hypothesis can be rejected. The putative enhancer was found specifically activated and accessible in 9 out of 9 salvage-dependent cancer models, but not (0 of 9) in the PH-amplified cancers or normal cell models.

### CRISPR Interference-Mediated Repression of the Enhancer Region.

CRISPR interference dCas9 sgRNAs were identified using the MIT CRISPR Design tool, and control, non-targeting sgRNAs were selected from the GeCKOv2 library. All sgRNA sequences are listed in the Sequence Listing provided herewith. For repression of the NAMPT enhancer, lenti-KRAB-dCas9-blast was provided by M. Meyerson and X. Zhang [See, for example, Ref 33]. sgRNAs were cloned into lentiGuide-Puro using BsmBI as the restriction site (Addgene, 52963). Salvage dependent H460^{Sal-dep}, U87^{Sal-dep}, and HCT116^{Sal-dep} cancer cells were first infected with lenti-KRAB-dCas9-blast and selected with 6 μg ml⁻¹ blasticidin. Cells stably expressing KRAB-dCas9 were then subsequently infected with sgRNAs and selected with 2 μg ml⁻¹ puromycin.

### ChIP-qPCR.

ChIP experiments were performed according to the manufacturer's instructions (CST, D1K6D 9004). In brief, around 1-2×10⁷ cells were crosslinked with 1% formaldehyde for 10 min at room temperature. Cells were washed twice in ice-cold PBS, scraped in ice-cold PBS with protease inhibitor cocktail and collected as pellets after centrifugation at 2,000 g for 5 min. at 4° C. Nuclei pellet was incubated with micrococcal nuclease for 20 min. at 37° C. with frequent mixing to digest DNA to a length of approximately 150-900 bp. Samples were sonicated to break the nuclear membrane and centrifuged at 9,400 g for 10 min. at 4° C. to clarify lysates. Chromatin samples were treated with RNaseA (5 μg ml⁻¹) and protease K (0.2 mg ml⁻¹) and purified using DNA purification spin columns. For chromatin immunoprecipitation 5-10 μg of digested, cross-linked chromatin per immunoprecipitation was used. Positive control histone H3 (CST, D2B12 4620) and negative control normal rabbit IgG (CST, 2729 samples) was included in all ChIP-qPCR experiments to test for the enrichment of RPL30 promoter. Sonicated chromatin samples were incubated with 5 μg H3K27ac antibody (active motif, 39133) or respective controls at 4° C. with rotation. After overnight incubation, protein G Magnetic beads were added to the ChIP reactions and incubated for an additional 4 h at 4° C. with rotation to collect the immunoprecipitated chromatin. Magnetic beads were subsequently washed twice, each with 1 ml of low- to high-salt buffer. The chromatin was eluted in ChIP elution buffer upon incubation of magnetic beads at 65° C. for 30 min with gentle mixing. Input and chromatin samples were treated with 0.1 M NaCl, Proteinase K (0.2 mg ml⁻¹), RNaseA (5 μg ml⁻¹) and reverse cross-linked at 65° C. overnight followed up with purification using DNA purification spin columns. qPCR was performed on the purified ChIP DNA using iQ SYBR Green Supermix (Bio-Rad) on the CFX96 Touch Real Time PCR Detection system (Bio-Rad) following the manufacturer's instructions. Enhancer occupancy was calculated as percentage of input DNA using the ΔΔCt method. Primer sequences are listed in Sequence Listing provided herewith.

### Luciferase Assay.

Cells were transfected at 70% confluence using Lipofectamine2000 Reagent (Thermo Fisher Scientific), according to the manufacturer's guidelines. In all transfections the pRL-TK Renilla reporter vector (Promega) was used as an internal control. Cells were lysed, and Renilla and firefly luciferase activities were measured using the Dual-Luciferase Reporter Assay System (Promega) and a Tecan Infinite M1000 microplate reader. Luciferase activities were normalized to Renilla internal control luminescence.

### Xenografts.

All procedures were reviewed and approved by the Institutional Animal Use and Care Committee (IACUC) at University of California, San Diego. Tumor size must not exceed 20 mm at the largest diameter, and this tumor threshold was never exceeded in any experiment. Five-six-week-old female athymic nu/nu mice were purchased from Harlan Sprague Dawley. Subcutaneous tumors were established by injection of 5×10⁵ parental or genetically engineered lung cancer H460 cells, and 3×10⁶ parental and 2.5×10⁶ genetically engineered OV4 cells, implanted subcutaneously into the flanks of immune-compromised female mice. Genetically engineered OV4PH-amp cells stably expressing DOX-inducible shRNA against NAPRT (ishNAPRT), NADSYN1 (ishNADSYN1), NAMPT (ishNAMPT), or NMRK1 (ishNMRK1) were independently inoculated at the left flank, and H460^{Sal-dep} cells stably expressing DOX-inducible shRNA against NAPRT (ishNAPRT), NAMPT (ishNAMPT), NMRK1 (ishNMRK1), or both NAMPT and NMRK1 (ishNAMPT+ishNMRK1) were independently inoculated at the right flank in individual mice. To test for the rescue of the knockdown phenotype, OV4PH-amp or H460^{Sal-dep} cells stably expressing DOX-inducible shRNA targeting the 3′ UTR of the target genes, NAPRT (ishNAPRT), NAMPT (ishNAMPT), or NMRK1 (ishNMRK1), or co-deletion of NAMPT and NMRK1 (ishNAMPT+NMRK1) were subcutaneously implanted at the left flank in individual mice. The same clone of stably engineered OV4PH-amp or H460^{Sal-dep} cells but with expression of exogenous cDNA corresponding to the target not susceptible to silencing compared to the endogenous copy, (ishNAPRT(+naprt−Flag)), (ishNAMPT(+nampt−Flag)), or (ishNAMPT+NMRK1(+nmrk1−Flag)) were subcutaneously implanted at the right flank in individual mice. ishNTC was used as a non-targeting control inducible shRNA for both tumor types. Each cohort categorized for both tumor types consisted of eight mice. After implantation of tumor cells, mice were randomized and then allocated into cages. Mice were fed DOX-containing chow (200 mg kg⁻¹, Bio-Serv), starting 7 days after implantation until the end of the experiment. For FK-866 treatment, tumor-bearing mice were intraperitoneally injected with FK-866 at 5 mg kg⁻¹, or 20 mg kg⁻¹, or with PBS twice daily for 5 days per week for 3 weeks. For NADSYN1i treatment, tumor-bearing mice were intraperitoneally injected with NADSYN1i at 10 mg kg⁻¹ or 30 mg kg⁻¹ or with PBS once daily for 2 weeks. Tumor volume was monitored every 3-5 days over a 30-day period using electronic calipers. Tumor volumes were calculated by caliper measurements of the short (a) and long (b) tumor diameters (volume=a2×b/2) or of tumor height (h), short (a) and long (b) tumor width (volume=h×a×b/2) depending on tumor shape (mean tumor volume±s.e.m.). In vivo tumor models do not mimic human physiology, considering the bioavailability of NAD precursors. In the mouse DOX diet, only niacin (86.9 mg kg⁻¹) and TRP (2.5 g kg⁻¹) were present, and the availability of nicotinic acid and nicotinamide riboside was unique and only orally bioavailable. Therefore, to mimic human physiology and pathophysiology where there is a ubiquitous presence of all the NAD precursors, nicotinamide, nicotinamide riboside, nicotinic acid, and TRP were orally provided in the drinking water ad libitum (200 mg kg⁻¹ day⁻¹) to nude mice bearing tumors

### Immunohistochemistry and Tissue Ki67 Staining.

Formalin-fixed, paraffin-embedded (FFPE) tissue sections were prepared by the Histology Core Facility at UCSD Moores Cancer Center. Immunohistochemistry was performed according to standard procedures. Antigen was retrieved by boiling slides in 0.01 M of sodium citrate (pH 6.0) in a microwave for 15 min. Sections were incubated with primary Ki67 antibody (Thermo Fisher Scientific, PA5-16785) at 4° C. overnight, followed by incubation with biotinylated secondary antibodies at room temperature for 30 min. Three representative images from each immunostained section were captured using DP 25 camera mounted on an Olympus BX43 microscope at 40× magnification. Quantitative analysis of the IHC images was performed using image analyzing software (Visiopharm).

### Tissue TUNEL Staining.

Cell death analysis was conducted in xenografts using the In situ Cell Death Detection kit, Fluorescein (Sigma-Aldrich) according to the manufacturer's protocol. In brief, paraffin embedded tissues were deparaffinized with xylene and rehydrated with ethanol, followed by treatment with proteinase K (5 μg ml-1, New England Biolabs). Sections were then incubated with reaction buffer for 1 h at 37° C. in a humidified chamber with no access to light. DAPI was used to stain DNA. Images were acquired with an Olympus IX81 microscope. Three representative images from each section were captured using DP 25 camera mounted on an Olympus BX43 microscope at 40× magnification. Quantitative analysis of the images detecting TUNEL+ nuclei was performed using ImageJ software (NIH).

### Statistics and Reproducibility.

Graphpad Prism software was used to conduct the statistical analysis of all data. No statistical methods were used to predetermine sample size. For xenograft experiments, female athymic nu/nu mice injected with tumor cells were randomized before being allocated to respective cages. All other experiments conducted for the study were not randomized, and the investigators were not blinded to either allocation during experiments or to outcome assessments. Variation in data in all experiments is presented as mean±s.d. except for variation for xenograft tumor volume, which is illustrated as the scanning electron micrograph. Some quantitative results were assessed by unpaired Student's t-test after confirming that the data met appropriate assumptions. The Student's t-test assumed two-tailed distributions to calculate statistical significance between groups. For comparisons among three or more groups, statistical significance was assessed using one-way ANOVA followed by Tukey's multiple comparisons test. To examine significance in xenograft tumor growth between two or among three or more groups, statistical significance was assessed using two-way ANOVA, to calculate significance on repeated measurements over time followed by Tukey's multiple comparisons test. As required for ANOVA, homogeneity of variance was tested among the groups using the Brown-Forsythe test. In all xenograft assays, subcutaneous tumors were established in 5-6-week-old female athymic nu/nu mice and randomized into different cohorts. Each categorized cohort for both tumor-types consisted of eight mice.

### EMBODIMENTS

The embodiments described below are non-limiting embodiments.

Embodiment 1. A method for treating a NAD salvage pathway-dependent cancer in a subject in need thereof, the method comprising:

(i) selecting a subject with a NAD salvage pathway dependent cancer;
(ii) administering to the subject a therapeutically effective amount of an anticancer agent.

Embodiment 2. The method of embodiment 1, wherein the anticancer agent is a NAMPT inhibitor.

Embodiment 3. The method of embodiment 2, wherein said NAMPT inhibitor is FK866.

Embodiment 4. The method of embodiment 2 or 3, wherein said NAMPT inhibitor is administered at a low therapeutically effective amount.

Embodiment 5. The method of embodiment 1 or 2, further comprising administering a NMRK1 enzyme pathway modulator.

Embodiment 6. A method for treating cancer in a subject in need thereof, said method comprising:

(i) selecting a subject having a cancer with an amplified Preiss Handler pathway;
(ii) administering to the subject a therapeutically effective amount of an anticancer agent.

Embodiment 7. The method of embodiment 6, wherein the anticancer agent is an inhibitor of NADSYN1.

Embodiment 8. The method of embodiment 7, wherein the inhibitor of NADSYN1 is (N-(3,4-dichlorophenyl)-4-{[(4-nitrophenyl)carbamoyl]amino}benzenesulfonamide).

Embodiment 9. The method of embodiment 6, wherein the anticancer agent is an inhibitor of NAPRT.

Embodiment 10. The method of embodiment 9, wherein the inhibitor of NAPRT is 2-hydroxynicotinic acid.

Embodiment 11. A method for treating cancer in a subject in need thereof, said method comprising administering to the subject a therapeutically effective amount of an anticancer agent, wherein the subject has an amplified Preiss Handler pathway gene.

Embodiment 12. The method of embodiment 11, comprising selecting a subject having cancer prior to administering said anticancer agent.

Embodiment 13. The method of any one of embodiments 11-12, wherein the cancer is selected from ovarian cancer, prostate cancer, esophageal cancer, salivary gland cancer, breast cancer, liver cancer, pancreatic cancer, stomach cancer, lung cancer, bladder cancer, colon cancer and uterine cancer.

Embodiment 14. The method of any one of embodiments 11-13, wherein the amplified Preiss Handler pathway gene is an amplified NAPRT gene or an amplified NADSYN1 gene.

Embodiment 15. The method of any one of embodiments 11-14, wherein the anticancer agent is an NAPRT inhibitor or an NADSYN1 inhibitor.

Embodiment 16. The method of embodiment 15, wherein the inhibitor is an antibody, an antisense nucleic acid, a gene editing reagent or a small molecule.

Embodiment 17. The method of any one of embodiments 15-16, wherein the anticancer agent is an NAPRT inhibitor.

Embodiment 18. The method of embodiment 17, wherein the NAPRT inhibitor is 2-hydroxynicotinic acid.

Embodiment 19. The method of any one of embodiments 15-16, wherein the anticancer agent is an NADSYN1 inhibitor.

Embodiment 20. The method of embodiment 19, wherein the NADSYN1 inhibitor is (N-(3,4-dichlorophenyl)-4-{[(4-nitrophenyl)carbamoyl]amino}benzenesulfonamide)

Embodiment 21. A method for treating cancer in a subject in need thereof, the method comprising administering to the subject a therapeutically effective amount of an anticancer agent, wherein the subject has a NAD salvage pathway-dependent cancer.

Embodiment 22. The method of embodiment 21, comprising selecting a subject having cancer prior to administering said anticancer agent.

Embodiment 23. The method of embodiment 21 or 22, wherein the cancer is selected from muscle cancer, brain cancer, lymph node cancer, thyroid cancer, kidney cancer, and adrenal gland cancer.

Embodiment 24. The method of any one of embodiments 21-23, wherein the anticancer agent is a NAMPT inhibitor or a NMRK1 inhibitor.

Embodiment 25. The method of embodiment 24, wherein the inhibitor is an antibody, an antisense nucleic acid, a gene editing reagent or a small molecule.

Embodiment 26. The method of any one of embodiments 21-25, wherein the anticancer agent is a NAMPT inhibitor.

Embodiment 27. The method of embodiment 26, wherein the NAMPT inhibitor is FK866 (N-[4-(1-benzoyl-4-piperidinyl)butyl]-3-(3-pyridinyl)-2E-propenamide).

Embodiment 28. The method of embodiment 21, wherein the anticancer agent is a NAMPT enzyme pathway modulator.

Embodiment 29. The method of embodiment 28, wherein the modulator is an NAMPT enhancer region inhibitor.

Embodiment 30. The method of any one of embodiments 24-29, wherein the NAMPT inhibitor is administered at a low therapeutically effective amount.

Embodiment 31. The method of any one of embodiments 21-25, wherein the anticancer agent is a NMPRK1 inhibitor.

Embodiment 32. The method of any one of embodiments 21-25, wherein the method comprises administering a NAMPT inhibitor and a NMRK1 inhibitor.

Embodiment 33. A method of detecting NAPRT gene expression or NADSYN1 gene expression in a subject that has cancer, the method comprising measuring in a biological sample from the patient, a DNA copy number of a gene selected from NAPRT and NADSYN1.

Embodiment 34. The method of embodiment 33, further comprising classifying the cancer as a Preiss Handler pathway dependent cancer if the DNA copy number is amplified above a threshold.

Embodiment 35. The method of embodiment 33 wherein the gene is NAPRT.

Embodiment 36. The method of embodiment 33, wherein the gene is NADSYN1.

Embodiment 37. The method of any one of embodiments 33-36, wherein the threshold is a copy number of 4 or more.

Embodiment 38. The methods of any one of claims embodiments 33-37, further comprising administering an anticancer agent.

Embodiment 39. The method of embodiment 37, wherein the anticancer agent is an NAPRT inhibitor or an NADSYN1 inhibitor.

Embodiment 40. The method of claim embodiment 38, wherein the inhibitor is an antibody, an antisense nucleic acid, a gene editing reagent or a small molecule.

Embodiment 41. The method of any one of embodiments 38-40, wherein the anticancer agent is an NAPRT inhibitor.

Embodiment 42. The method of embodiment 41, wherein the NAPRT inhibitor is 2-hydroxynicotinic acid.

Embodiment 43. The method of any one of embodiments 38-40, wherein the anticancer agent is an NADSYN1 inhibitor.

Embodiment 44. The method of embodiment 43, wherein the NADSYN1 inhibitor is (N-(3,4-dichlorophenyl)-4-{[(4-nitrophenyl)carbamoyl]amino}benzenesulfonamide).

Embodiment 45. The method of embodiment 33, further comprising classifying the cancer as a NAMPT salvage pathway cancer if the DNA copy number is selected from 0, 1, and 2.

Embodiment 46. The method of embodiment 45, further comprising administering an anticancer agent.

Embodiment 47. The method of embodiment 46, wherein the anticancer agent is a NAMPT inhibitor or a NMRK1 inhibitor.

Embodiment 48. The method of embodiment 47, wherein the inhibitor is an antibody, an antisense nucleic acid, a gene editing reagent or a small molecule.

Embodiment 49. The method of any one of embodiment 45-48, wherein the anticancer agent is a NAMPT inhibitor.

Embodiment 50. The method of embodiment 49, wherein the NAMPT inhibitor is FK866 (N-[4-(1-benzoyl-4-piperidinyl)butyl]-3-(3-pyridinyl)-2E-propenamide).

Embodiment 51. The method of any one of embodiment 45-48, wherein the anticancer agent is a NAMPT enzyme pathway modulator.

Embodiment 52. The method of embodiment 51, wherein the modulator is an NAMPT enhancer region inhibitor.

Embodiment 53. The method of any one of embodiments 47-50, wherein the NAMPT inhibitor is administered at a low therapeutically effective amount.

Embodiment 54. The method of any one of embodiments 46-48, wherein the anticancer agent is a NMPRK1 inhibitor.

Embodiment 55. The method of any one of embodiments 46-48, wherein the method comprises administering a NAMPT inhibitor and a NMRK1 inhibitor.

Embodiment 56. A method for treating cancer in a subject in need thereof, said method comprising administering to the subject a therapeutically effective amount of an anticancer agent, wherein the subject has a Preiss Handler pathway-dependent tumor.

Embodiment 57. The method of embodiment 56, comprising selecting a subject having cancer prior to administering said anticancer agent.

Embodiment 58. The method of any one of embodiments 56-57, wherein the cancer is selected from the group consisting of ovarian cancer, prostate cancer, esophageal cancer, salivary gland cancer, breast cancer, liver cancer, pancreatic cancer, stomach cancer, lung cancer, bladder cancer, colon cancer, and uterine cancer.

Embodiment 59. The method of any one of embodiments 56-68, wherein the Preiss Handler pathway dependence is the result of an amplified NAPRT gene or gene product thereof, or an amplified NADSYN1 gene or gene product thereof.

Embodiment 60. The method of any one of embodiments 56-59, wherein the anticancer agent is a NAPRT inhibitor or a NADSYN1 inhibitor.

Embodiment 61. The method of embodiment 60, wherein the inhibitor is an antibody, an antisense nucleic acid, a gene editing reagent, or a small molecule.

Embodiment 62. The method of any one of embodiments 60-61, wherein the anticancer agent is a NAPRT inhibitor.

Embodiment 63. The method of embodiment 62, wherein the NAPRT inhibitor is 2-hydroxynicotinic acid.

Embodiment 64. The method of any one of embodiments 60-61, wherein the anticancer agent is a NADSYN1 inhibitor.

Embodiment 65. The method of embodiment 64, wherein the NADSYN1 inhibitor is (N-(3,4-dichlorophenyl)-4-{[(4-nitrophenyl)carbamoyl]amino}benzenesulfonamide).

Embodiment 66. A composition comprising a NAMPT inhibitor and a NMRK1 inhibitor.

Embodiment 67. The composition of embodiment 66, wherein the NAMPT inhibitor is an antibody, an antisense nucleic acid, a gene editing reagent, or a small molecule.

Embodiment 68. The composition of embodiment 67, wherein the NAMPT inhibitor is FK866 (N-[4-(1-benzoyl-4-piperidinyl)butyl]-3-(3-pyridinyl)-2E-propenamide).

Embodiment 69. The composition of embodiment 66, wherein the NAMPT inhibitor is a NAMPT enzyme pathway modulator.

Embodiment 70. The composition of embodiment 69, wherein the modulator is a NAMPT enhancer region inhibitor.

Embodiment 71. The composition of embodiment 66, wherein the NMRK1 inhibitor is an antibody, an antisense nucleic acid, a gene editing reagent, or a small molecule.

Embodiment 72. The composition of embodiment 66, wherein the NMRK1 inhibitor is a NMRK1 enzyme pathway modulator.

Embodiment 73. A pharmaceutical composition comprising the composition of any of embodiments 66 to 72 and a pharmaceutically acceptable excipient.

### REFERENCES

1. Roychowdhury, S. & Chinnaiyan, A. M. Translating genomics for precision cancer medicine. Annu. Rev. Genomics Hum. Genet. 15, 395-415 (2014).
2. Mayers, J. R. & Vander Heiden, M. G. Nature and nurture: what determines tumor metabolic phenotypes? Cancer Res. 77, 3131-3134 (2017).
3. Cantó, C., Menzies, K. J. & Auwerx, J. NAD⁺ metabolism and the control of energy homeostasis: a balancing act between mitochondria and the nucleus. Cell Metab. 22, 31-53 (2015).
4. Verdin, E. NAD⁺ in aging, metabolism, and neurodegeneration. Science 350, 1208-1213 (2015).
5. Vander Heiden, M. G. & DeBerardinis, R. J. Understanding the intersections between metabolism and cancer biology. Cell 168, 657-669 (2017).
6. Pavlova, N. N. & Thompson, C. B. The Emerging hallmarks of cancer metabolism. Cell Metab. 23, 27-47 (2016).
7. Liu, L. et al. Quantitative analysis of NAD synthesis-breakdown fluxes. Cell Metab. 27, 1067-1080.e5 (2018).
8. Chiarugi, A., Dölle, C., Felici, R. & Ziegler, M. The NAD metabolome—a key determinant of cancer cell biology. Nat. Rev. Cancer 12, 741-752 (2012).
9. Ryu, K. W. et al. Metabolic regulation of transcription through compartmentalized NAD⁺ biosynthesis. Science 360, eaan5780 (2018).
10. Kaelin, W. G. Jr & McKnight, S. L. Influence of metabolism on epigenetics and disease. Cell 153, 56-69 (2013).
11. Bogan, K. L. & Brenner, C. Nicotinic acid, nicotinamide, and nicotinamide riboside: a molecular evaluation of NAD⁺ precursor vitamins in human nutrition. Annu. Rev. Nutr. 28, 115-130 (2008).
12. Katsyuba E. et al. De novo NAD⁺ synthesis enhances mitochondrial function and improves health. Nature 563, 354-359 (2018).
13. Tateishi, K. et al. Extreme vulnerability of IDH1 mutant cancers to NAD⁺ depletion. Cancer Cell 28, 773-784 (2015).
14. Piacente, F. et al. Nicotinic acid phosphoribosyltransferase regulates cancer cell metabolism, susceptibility to NAMPT inhibitors, and DNA repair. Cancer Res. 77, 3857-3869 (2017).
15. Hnisz, D. et al. Super-enhancers in the control of cell identity and disease. Cell 155, 934-947 (2013).
16. Leung, D. et al. Integrative analysis of haplotype-resolved epigenomes across human tissues. Nature 518, 350-354 (2015).
17. Gilbert, L. A. et al. CRISPR-mediated modular RNA-guided regulation of transcription in eukaryotes. Cell 154, 442-451 (2013).
18. Frederick, D. W. et al. Loss of NAD homeostasis leads to progressive and reversible degeneration of skeletal muscle. Cell Metab. 24, 269-282 (2016).
19. Moro, W. B., Yang, Z., Kane, T. A., Brouillette, C. G. & Brouillette, W. J. Virtual screening to identify lead inhibitors for bacterial NAD synthetase (NADs).Bioorg. Med. Chem. Lett. 19, 2001-2005 (2009).
20. Devedjiev, Y. et al. Stabilization of active-site loops in NH3-dependent NAD⁺ synthetase from Bacillus subtilis. Acta Crystallogr. D 57, 806-812 (2001).
21. Hara, N. et al. Molecular identification of human glutamine- and ammonia dependent NAD synthetases. Carbon-nitrogen hydrolase domain confers glutamine dependency. J. Biol. Chem. 278, 10914-10921 (2003).
24. Heintzman, N. D. et al. Distinct and predictive chromatin signatures of transcriptional promoters and enhancers in the human genome. Nat. Genet. 39, 311-318 (2007).
25. Cerami, E. et al. The cBio cancer genomics portal: an open platform for exploring multidimensional cancer genomics data. Cancer Discov. 2, 401-404 (2012).
26. Lonsdale, J. et al. The Genotype-Tissue Expression (GTEx) project. Nat. Genetics 45, 580-585 (2013).
27. Grossman, R. L. et al. Toward a shared vision for cancer genomic data. N. Engl. J. Med. 375, 1109-1112 (2016).
28. Hartigan, J. A. & Hartigan, P. M. The dip test of unimodality. Ann. Stat. 13, 70-84 (1985).
29. Hasmann, M. & Schemainda, I. FK866, a highly specific noncompetitive inhibitor of nicotinamide phosphoribosyltransferase, represents a novel mechanism for induction of tumor cell apoptosis. Cancer Res. 63, 7436-7442 (2003).
30. Turner, K. M. et al. Extrachromosomal oncogene amplification drives tumor evolution and genetic heterogeneity. Nature 543, 122-125 (2017).
31. Wojcik, M., Seidle, H. F., Bieganowski, P. & Brenner, C. Glutamine-dependent NAD+ synthetase. How a two-domain, three-substrate enzyme avoids waste. J. Biol. Chem. 281, 33395-33402 (2006).
32. Zalkin, H. NAD synthetase. Methods Enzymol. 113, 297-302 (1985).
33. Zhang, X. et al. Identification of focally amplified lineage-specific super-enhancers in human epithelial cancers. Nat. Genet. 48, 176-182 (2016).

What is claimed is:

1. A method for treating cancer in a subject in need thereof, said method comprising administering to the subject a therapeutically effective amount of an anticancer agent, wherein the subject has an amplified Preiss Handler pathway gene.

2. The method of claim 1, comprising selecting a subject having cancer prior to administering said anticancer agent.

3. The method of claim 1, wherein the cancer is ovarian cancer, prostate cancer, esophageal cancer, salivary gland cancer, breast cancer, liver cancer, pancreatic cancer, stomach cancer, lung cancer, bladder cancer, colon cancer, or uterine cancer.

4. The method of any one of claim 1, wherein the amplified Preiss Handler pathway gene is an amplified NAPRT gene or an amplified NADSYN1 gene.

5. The method of claim 1, wherein the anticancer agent is a NAPRT inhibitor or a NADSYN1 inhibitor.

6. The method of claim 5, wherein the inhibitor is an antibody, an antisense nucleic acid, a gene editing reagent, or a small molecule.

7. The method of any one of claim 5, wherein the anticancer agent is a NAPRT inhibitor.

8. The method of claim 7, wherein the NAPRT inhibitor is 2-hydroxynicotinic acid.

9. The method of any one of claim 5, wherein the anticancer agent is a NADSYN1 inhibitor.

10. The method of claim 9, wherein the NADSYN1 inhibitor is (N-(3,4-dichlorophenyl)-4-{[(4-nitrophenyl)carbamoyl]amino}benzenesulfonamide).

11. A method for treating cancer in a subject in need thereof, the method comprising administering to the subject a therapeutically effective amount of an anticancer agent, wherein the subject has a NAD salvage pathway-dependent cancer.

12. The method of claim 11, comprising selecting a subject having cancer prior to administering said anticancer agent.

13. The method of claim 11, wherein the cancer is selected from the group consisting of muscle cancer, brain cancer, lymph node cancer, thyroid cancer, kidney cancer, and adrenal gland cancer.

14. The method of claim 11, wherein the anticancer agent is a NAMPT inhibitor or a NMRK1 inhibitor.

15. The method of claim 14, wherein the inhibitor is an antibody, an antisense nucleic acid, a gene editing reagent, or a small molecule.

16. The method of claim 11, wherein the anticancer agent is a NAMPT inhibitor.

17. The method of claim 16, wherein the NAMPT inhibitor is FK866 (N-[4-(1-benzoyl-4-piperidinyl)butyl]-3-(3-pyridinyl)-2E-propenamide).

18. The method of claim 11, wherein the anticancer agent is a NAMPT enzyme pathway modulator.

19. The method of claim 18, wherein the modulator is a NAMPT enhancer region inhibitor.

20. The method of claim 14, wherein the NAMPT inhibitor is administered at a low therapeutically effective amount.

21. The method of claim 11, wherein the anticancer agent is a NMPRK1 inhibitor.

22. The method of claim 11 wherein the method comprises administering a NAMPT inhibitor and a NMRK1 inhibitor.

23. A method of detecting NAPRT gene expression or NADSYN1 gene expression in a subject that has cancer, the method comprising measuring in a biological sample from the subject, a DNA copy number, RNA expression, or protein expression, of a gene selected from NAPRT and NADSYN1.

24. The method of claim 23, further comprising classifying the cancer as a Preiss Handler pathway-dependent cancer if the DNA copy number is amplified above a threshold.

25. The method of claim 23 wherein the gene is NAPRT.

26. The method of claim 23, wherein the gene is NADSYN1.

27. The method of claim 23, wherein the threshold is a copy number of 4 or more.

28. The methods of claim 23, further comprising administering an anticancer agent.

29. The method of claim 28, wherein the anticancer agent is a NAPRT inhibitor or a NADSYN1 inhibitor.

30. The method of claim 29, wherein the inhibitor is an antibody, an antisense nucleic acid, a gene editing reagent, or a small molecule.

31. The method of claim 28, wherein the anticancer agent is a NAPRT inhibitor.

32. The method of claim 31, wherein the NAPRT inhibitor is 2-hydroxynicotinic acid.

33. The method of claim 28, wherein the anticancer agent is a NADSYN1 inhibitor.

34. The method of claim 33, wherein the NADSYN1 inhibitor is (N-(3,4-dichlorophenyl)-4-{[(4-nitrophenyl)carbamoyl]amino}benzenesulfonamide).

35. The method of claim 23, further comprising classifying the cancer as a NAMPT salvage pathway cancer if the DNA copy number is selected from 0, 1, and 2.

36. The method of claim 35, further comprising administering an anticancer agent.

37. The method of claim 36, wherein the anticancer agent is a NAMPT inhibitor or a NMRK1 inhibitor.

38. The method of claim 37, wherein the inhibitor is an antibody, an antisense nucleic acid, a gene editing reagent, or a small molecule.

39. The method of claim 35, wherein the anticancer agent is a NAMPT inhibitor.

40. The method of claim 39, wherein the NAMPT inhibitor is FK866 (N-[4-(1-benzoyl-4-piperidinyl)butyl]-3-(3-pyridinyl)-2E-propenamide).

41. The method of claim 35, wherein the anticancer agent is a NAMPT enzyme pathway modulator.

42. The method of claim 41, wherein the modulator is a NAMPT enhancer region inhibitor.

43. The method of claim 37, wherein the NAMPT inhibitor is administered at a low therapeutically effective amount.

44. The method of claim 34, wherein the anticancer agent is a NMPRK1 inhibitor.

45. The method of claim 34, wherein the method comprises administering a NAMPT inhibitor and a NMRK1 inhibitor.

46. A method for treating cancer in a subject in need thereof, said method comprising administering to the subject a therapeutically effective amount of an anticancer agent, wherein the subject has a Preiss Handler pathway-dependent tumor.

47. The method of claim 46, comprising selecting a subject having cancer prior to administering said anticancer agent.

48. The method of claim 46, wherein the cancer is selected from the group consisting of ovarian cancer, prostate cancer, esophageal cancer, salivary gland cancer, breast cancer, liver cancer, pancreatic cancer, stomach cancer, lung cancer, bladder cancer, colon cancer, and uterine cancer.

49. The method of claim 46, wherein the Preiss Handler pathway dependence is the result of an amplified NAPRT gene or gene product thereof, or an amplified NADSYN1 gene or gene product thereof.

50. The method of any claim 46, wherein the anticancer agent is a NAPRT inhibitor or a NADSYN1 inhibitor.

51. The method of claim 50, wherein the inhibitor is an antibody, an antisense nucleic acid, a gene editing reagent, or a small molecule.

52. The method of claim 50, wherein the anticancer agent is a NAPRT inhibitor.

53. The method of claim 52, wherein the NAPRT inhibitor is 2-hydroxynicotinic acid.

54. The method of claim 50, wherein the anticancer agent is a NADSYN1 inhibitor.

55. The method of claim 54, wherein the NADSYN1 inhibitor is (N-(3,4-dichlorophenyl)-4-{[(4-nitrophenyl)carbamoyl]amino}benzenesulfonamide).

56. A composition comprising a NAMPT inhibitor and a NMRK1 inhibitor.

57. The composition of claim 56, wherein the NAMPT inhibitor is an antibody, an antisense nucleic acid, a gene editing reagent, or a small molecule.

58. The composition of claim 57, wherein the NAMPT inhibitor is FK866 (N-[4-(1-benzoyl-4-piperidinyl)butyl]-3-(3-pyridinyl)-2E-propenamide).

59. The composition of claim 56, wherein the NAMPT inhibitor is a NAMPT enzyme pathway modulator.

60. The composition of claim 59, wherein the modulator is a NAMPT enhancer region inhibitor.

61. The composition of claim 56, wherein the NMRK1 inhibitor is an antibody, an antisense nucleic acid, a gene editing reagent, or a small molecule.

62. The composition of claim 56, wherein the NMRK1 inhibitor is a NMRK1 enzyme pathway modulator.

63. A pharmaceutical composition comprising the composition claim 56 and a pharmaceutically acceptable excipient.
